(19) United States
(12) Patent Application Publication
(10) Pub. No.: US 2021/0170394 A1
(43) Pub. Date: Jun. 10, 2021

(54) LIQUID FEEDING DEVICE

(71) Applicant: FUJIFILM Corporation, Tokyo (JP)

(72) Inventors: Takeshi HAMA, Ashigarakami-gun (JP); Noboru KOMORI, Ashigarakami-gun (JP); Yuki INOUE, Ashigarakami-gun (JP); Aya OUCHI, Ashigarakami-gun (JP)

(73) Assignee: FUJIFILM Corporation, Tokyo (JP)

(21) Appl. No.: 17/074,009

(22) Filed: Oct. 19, 2020

(30) Foreign Application Priority Data
Dec. 9, 2019 (JP) 2019-222330

(51) Int. Cl. B01L 3/00; G01N 33/543

(52) U.S. Cl. CPC B01L 3/5027; G01N 33/54326; B01L 2200/0605; B01L 2400/0478; B01L 2300/0864; B01L 2400/0481; B01L 2200/0642

(57) ABSTRACT

Provided is a liquid feeding device, including: a test container including a first accommodation portion, a second accommodation portion, and a third accommodation portion each accommodating a liquid, a first flow path connecting the first accommodation portion and the second accommodation portion to each other at respective upper end positions thereof, and a second flow path connecting the second accommodation portion and the third accommodation portion to each other at respective upper end positions thereof, in which at least a portion forming an upper wall surface of the second accommodation portion has flexibility; and a pressing machine which includes a first pressing portion and presses the portion forming the upper wall surface of the second accommodation portion at a position closer to the first flow path from a center in a liquid feeding direction towards an inside of the second accommodation portion from an outside by the pressing portion, in which the liquid accommodated in the second accommodation portion is fed to the third accommodation portion by pressing the portion forming the upper wall surface of the second accommodation portion with the pressing machine.

### CROSS-REFERENCE TO RELATED APPLICATIONS

The present application claims priority under 35 U.S.C. § 119 to Japanese Patent Application No. 2019-222330 filed on Dec. 9, 2019. Each of the above application is hereby expressly incorporated by reference, in its entirety, into the present application.

### BACKGROUND OF THE INVENTION

### 1. Field of the Invention

The technology of the present disclosure relates to a liquid feeding device.

### 2. Description of the Related Art

Test containers such as a test cartridge, an analysis chip, and the like used for performing various analysis with respect to a specimen extracted from a biological sample are known.

JP2007-101428A discloses a cartridge for a chemical treatment comprising a plurality of wells (liquid accommodation portions) accommodating a liquid and configured by stacking an elastic member comprising a plurality of recesses on one surface on a substrate so that the recesses face the substrate side, and a flow path connecting between the wells. JP2007-101428A discloses a method for rotating a roller while pressing the elastic member of a cartridge for elastic deformation of the elastic member, to cause pressing of a liquid in the elastically deformed well to move to an adjacent well via the flow path connected to the well.

JP2003-166910A discloses a liquid feeding mechanism which feeds a liquid filled in a liquid tank to a flow path connected to the liquid tank by changing a volume of the liquid tank (liquid accommodation portion) formed to surround a wall, and an analysis device comprising the liquid feeding mechanism.

### SUMMARY OF THE INVENTION

For example, in order to perform nucleic acid extraction and analysis, it is necessary to provide a test container comprising at least three accommodation portions from an upstream side to a downstream side in a liquid feeding direction, in which the liquid is fed so that a liquid return to the upstream accommodation portion does not occur, in a case where the liquid is fed from the accommodation portion in the middle of the three accommodation portions to the downstream accommodation portion side.

In JP2007-101428A, the liquid is fed to the downstream side in a state where the upstream side is crushed with respect to the accommodation portion containing the liquid and the flow path is blocked, and accordingly, the liquid return to the upstream side does not occur. However, in JP2007-101428A, it is premised that all the liquid accommodated in the upstream accommodation portion is moved to the downstream side. Accordingly, the aspect of proceeding to the next step while remaining the liquid in the upstream accommodation portion cannot be applied. Meanwhile, JP2003-166910A discloses only liquid feeding between two accommodation portions, and a method for preferentially feeding a liquid to the downstream side from an accommodation portion, of which flow paths are connected to both the upstream side and the downstream side is not considered.

In addition, in the test container disclosed in both JP2007-101428A and JP2003-166910A, the flow path connecting the liquid accommodation portions is disposed to connect lower ends of the liquid accommodation portions, and accordingly, even in a case where an external force is not applied, the liquid may pass the flow path and flow into the adjacent accommodation portion due to a capillary force or the like.

The technology of the disclosure is made in view of the above circumstance, and an object thereof is to provide a liquid feeding device including a test container, comprising at least three accommodation portions accommodating a liquid, and capable of preventing a liquid return to an upstream accommodation portion, in a case where the liquid accommodated in a middle accommodation portion of the three accommodation portions is fed to a downstream accommodation portion.

According to a first aspect of the disclosure, there is provided a liquid feeding device, comprising: a test container including a first accommodation portion, a second accommodation portion, and a third accommodation portion each accommodating a liquid, a first flow path connecting the first accommodation portion and the second accommodation portion to each other at respective upper end positions thereof, and a second flow path connecting the second accommodation portion and the third accommodation portion to each other at respective upper end positions thereof, in which at least a portion forming an upper wall surface of the second accommodation portion has flexibility; and

a pressing machine which includes a pressing portion and presses the portion forming the upper wall surface of the second accommodation portion at a position closer to the first flow path from a center in a liquid feeding direction towards an inside of the second accommodation portion from an outside by the pressing portion,

in which the liquid accommodated in the second accommodation portion is fed to the third accommodation portion by pressing the portion forming the upper wall surface of the second accommodation portion with the pressing machine.

According to a second aspect of the disclosure, there is provided a liquid feeding device, comprising: a test container including a first accommodation portion, a second accommodation portion, and a third accommodation portion each accommodating a liquid, a first flow path connecting the first accommodation portion and the second accommodation portion to each other at respective upper end positions thereof, and a second flow path connecting the second accommodation portion and the third accommodation portion to each other at respective upper end positions thereof, in which at least a first portion forming an upper wall surface of the first flow path and a second portion forming an upper wall surface of the second accommodation portion has flexibility; and

a pressing machine which includes a first pressing portion and a second pressing portion, presses the first portion forming the upper wall surface of the first flow path towards the inside of the first flow path from outside by the first pressing portion, and presses the second portion forming the upper wall surface of the second accommodation portion towards an inside of the second accommodation portion from an outside by the second pressing portion,

in which the liquid accommodated in the second accommodation portion is fed to the third accommodation portion by pressing the first portion and the second portion with the pressing machine.

In the liquid feeding device of the second aspect of the present disclosure, the pressing machine may simultaneously perform a pressing operation by the first pressing portion and a pressing operation by the second pressing portion.

In the liquid feeding device of the second aspect of the present disclosure, the pressing machine may perform a pressing operation by the first pressing portion before a pressing operation by the second pressing portion, and may perform the pressing operation by the second pressing portion while maintaining a pressing state by the first pressing portion.

In the liquid feeding device according to the first and second aspects of the present disclosure, the test container may include a main body portion in which a portion forming each of the first accommodation portion, the first flow path, the second accommodation portion, the second flow path, and the third accommodation portion is open, and an upper lid member including the portion forming the upper wall surface of the second accommodation portion, and the first accommodation portion, the first flow path, the second accommodation portion, the second flow path, and the third accommodation portion may be internally formed by covering the opening of the main body portion with the upper lid member.

In the liquid feeding device of the first and second aspects of the present disclosure, the upper lid member of the test container may have flexibility over an entire region.

In the liquid feeding device according to the first and second aspects of the present disclosure, the test container preferably comprises a liquid return prevention structure which prevents a backflow of the liquid to the first accommodation portion, in a case where the liquid accommodated in the second accommodation portion is fed to the third accommodation portion via the second flow path.

In the liquid feeding device according to the first and second aspects of the present disclosure, the liquid return prevention structure of the test container may have a structure in which a height from an inner bottom surface of the second accommodation portion to an inner bottom surface of the first flow path is higher than a height from the inner bottom surface of the second accommodation portion to an inner bottom surface of the second flow path.

In the liquid feeding device according to the first and second aspects of the disclosure, the liquid return prevention structure may have a structure of the first flow path and the second flow path in which a water contact angle of an inner surface of the first flow path is set to be greater than a water contact angle of an inner surface of the second flow path.

In the liquid feeding device according to the first and second aspects of the disclosure, the liquid return prevention structure may have a structure of a stepped portion which is provided between the first flow path and the second accommodation portion and which includes two or more steps from an inner bottom surface of the first accommodation portion.

In the liquid feeding device according to the first and second aspects of the present disclosure, the test container may further comprise a chromatographic carrier for performing a nucleic acid test; and a carrier accommodation portion accommodating the chromatographic carrier.

In the liquid feeding device according to the first and second aspects of the present disclosure, in the test container, the first accommodation portion may accommodate a first liquid containing magnetic particles, the first flow path allows separated magnetic particles separated from the first liquid to pass through the first flow path, and the second accommodation portion may accommodate the separated magnetic particles.

The liquid feeding device according to the first and second aspects of the present disclosure may further comprise a magnetic field generation and movement unit including a magnet and a movement mechanism which moves the magnet, the magnet may be disposed on the first accommodation portion of the test container to collect the magnet particles in the first liquid and the magnet is moved onto the second accommodation portion from the first accommodation portion along the first flow path, so that the separated magnetic particles separated from the first liquid passes through the first flow path to be moved to the second accommodation portion.

According to the liquid feeding device of the disclosure, in a liquid feeding device including a test container, comprising at least three accommodation portions accommodating a liquid, and capable of preventing a liquid return to an upstream accommodation portion, in a case where the liquid accommodated in a middle accommodation portion of the three accommodation portions is fed to a downstream accommodation portion.

### BRIEF DESCRIPTION OF THE DRAWINGS

FIG. 1 is a diagram showing a schematic configuration of a liquid feeding device 201.

FIG. 2 is an exploded perspective view showing a schematic configuration of a test container 211.

FIG. 3 is an cross-sectional view showing a schematic configuration of the test container 211.

FIG. 4 is a plan view showing a schematic configuration of a main body portion of the test container 211.

FIG. 5 is a diagram showing a liquid feeding method of the liquid feeding device 201.

FIG. 6 is a diagram showing a schematic configuration of a liquid feeding device 202.

FIG. 7 is a diagram showing a liquid feeding method of the liquid feeding device 202.

FIG. 8 is an exploded perspective view showing a schematic configuration of a test container 1.

FIG. 9 is an cross-sectional view showing a schematic configuration of the test container 1.

FIG. 10 is a plan view showing a schematic configuration of a main body portion of the test container 1.

FIG. 11 is an cross-sectional view showing a schematic configuration of a test container 1A of a modification example.

FIG. 12 is an exploded perspective view showing a schematic configuration of a test container 2.

FIG. 13 is an cross-sectional view showing a schematic configuration of the test container 2.

FIG. 14 is a plan view showing a schematic configuration of a main body portion of the test container 2.

FIG. 15 is an exploded perspective view showing a schematic configuration of a test container 3.

FIG. 16 is an cross-sectional view showing a schematic configuration of the test container 3.

FIG. 17 is a plan view showing a schematic configuration of a main body portion of the test container 3.

FIG. 18 is an cross-sectional view showing a schematic configuration of a test container 3A of a modification example.

FIG. 19 is an cross-sectional view showing a schematic configuration of a test container 4.

FIG. 20 is an cross-sectional view showing a schematic configuration of a test container 5.

FIG. 21 is an cross-sectional view showing a schematic configuration of a test container 6.

FIG. 22 is a schematic configuration diagram of a nucleic acid extraction test device 100.

FIG. 23 is an exploded perspective view of a test container and a diagram showing a main part of a dispenser.

FIG. 24 is a diagram showing a cross-sectional view of a test container and a magnet.

FIG. 25 is a diagram showing a cross-sectional view of a test container and a main part of a pressing machine.

FIG. 26 is a diagram for explaining pressed portions in the liquid feeding methods of examples and comparative examples.

### DESCRIPTION OF THE PREFERRED EMBODIMENTS

Hereinafter, an example of an embodiment according to the present invention will be described with reference to the drawings. A front direction, a rear direction, an upward direction, a downward direction, a left direction, and a right direction used in the description below correspond to “FR”, “RR”, “UP”, “DO”, “LH”, and “RH”, respectively, in the each drawing. Since these directions are defined for convenience of description, a device configuration is not limited to these directions. The FR side is an upstream side and the RR side is a downstream side in the use of a container. In addition, the scales and the like of the respective constituent elements in the drawings are suitably changed from the actual scales for the sake of easy visual recognition.

Liquid Feeding Device of First Embodiment

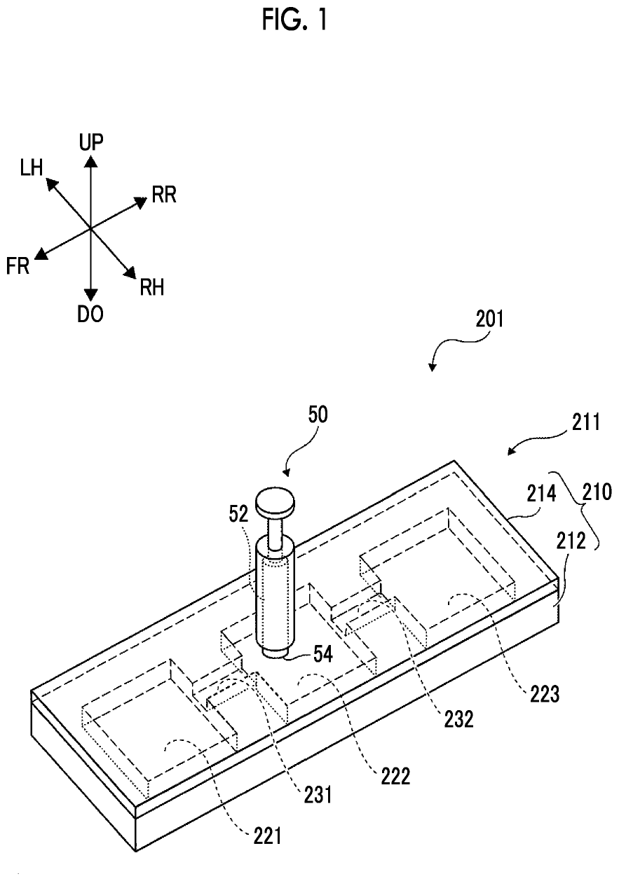

A liquid feeding device of the first embodiment will be described. FIG. 1 is a perspective view showing a schematic configuration of a liquid feeding device 201. The liquid feeding device 201 comprises a test container 211 and a pressing machine 50 having a plunger 52 as a pressing portion.

Test Container

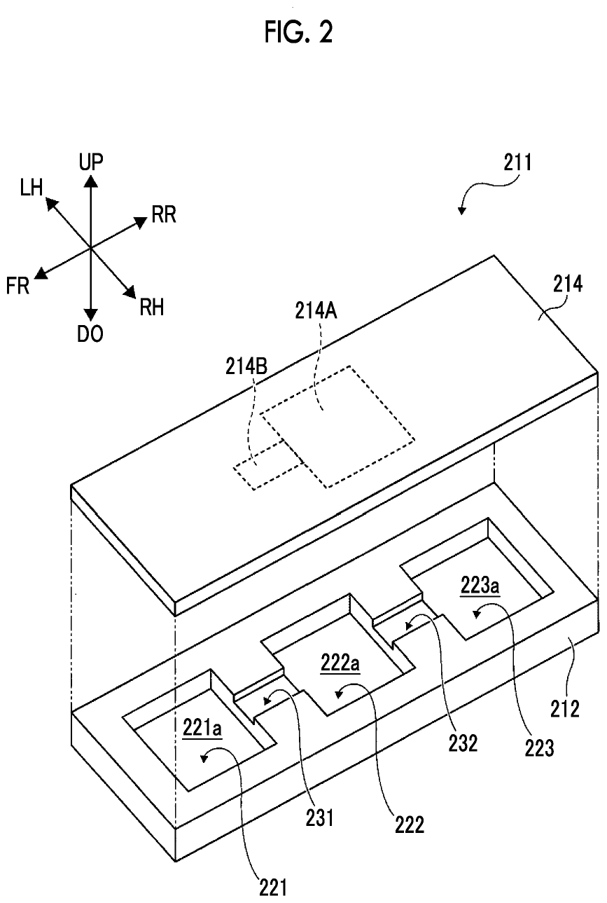

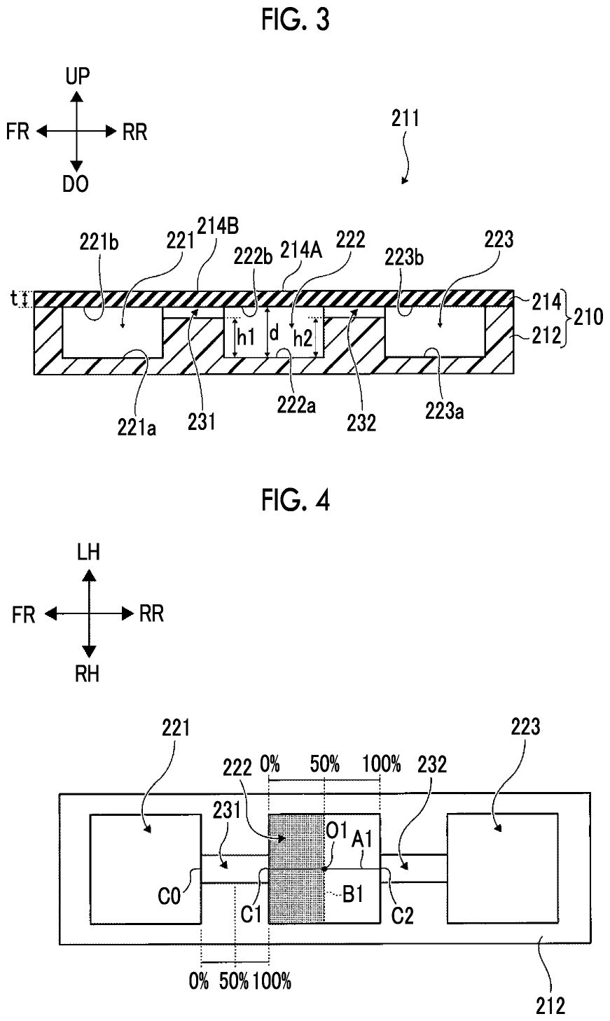

FIG. 2 is an exploded perspective view of the test container 211. FIG. 2 is an cross-sectional view showing a schematic configuration of the test container 211. FIG. 3 is a plan view showing a schematic configuration of a main body portion 212 of the test container 211.

The test container 211 comprises the container main body 210 including a first accommodation portion 221, a second accommodation portion 222, and a third accommodation portion 223 each accommodating a liquid, a first flow path 231 connecting the first accommodation portion 221 and the second accommodation portion 222 to each other at respective upper end positions thereof, and a second flow path 232 connecting the second accommodation portion 222 and the third accommodation portion 223 to each other at respective upper end positions thereof. The container main body 210 has flexibility so that it can be deformed toward the inside of the second accommodation portion 222 at least in the portion 214A forming the upper wall surface 222b of the second accommodation portion 222.

In this example, the container main body 210 comprises a main body portion 212 and an upper lid member 214. The main body portion 212 has an opening in a portion forming each of the first accommodation portion 221, the first flow path 231, the second accommodation portion 222, the second flow path 232, and the third accommodation portion 223. The container main body 210 has a configuration in which the first accommodation portion 221, the first flow path 231, the second accommodation portion 222, the second flow path 232, and the third accommodation portion 223 are formed therein by covering the opening of the main body portion 212 with the upper lid member 214. In other words, the container main body 210 configures the inner bottom surfaces 221a to 223a and the side wall surfaces of the accommodation portions 221 to 223, and the inner bottom surfaces 231a and 232a and the side wall surfaces of the flow paths 231 and 232, and the upper lid member 214 configures the upper wall surfaces 221b to 223b of the accommodation portions 221 to 223 and the upper wall surfaces 231b and 232b of the flow paths 231 and 232. However, the present invention is not limited to this configuration, as long as it has a configuration of comprising each accommodation portion and each flow path therein.

In this example, the upper lid member 214 has flexibility throughout. However, the entire upper lid member 214 does not have to be flexible, as long as the portion 214A configuring at least the upper wall surface 222b of the second accommodation portion 222 of the container main body 210, that is, the portion 214A of the upper lid member 214 has a flexible portion deformable in a direction toward the second accommodation portion 222.

The test container 211 comprises the first flow path 231 at the upper end position of the first accommodation portion 221 and the second accommodation portion 222, and the second flow path 232 at the upper end position of the second accommodation portion 222 and the third accommodation portion 223, respectively. Accordingly, the liquid accommodated in the accommodation portion is difficult to flow into the flow path, compared to a case where the flow path is comprised at a lower end or in the middle in a depth direction. Therefore, it is possible to prevent a passage of the liquid into the flow path due to a capillary phenomenon or the like without applying an external force. Meanwhile, since the portion 214A deformable toward the inside of the second accommodation portion 222 is comprised at the upper portion of the second accommodation portion 222, the portion 214A is deformed toward the inside of the second accommodation portion 222 to reduce a volume of the second accommodation portion 222, thereby easily realizing liquid feeding to the third accommodation portion 223 by pushing the liquid accommodated in the second accommodation portion 222.

Pressing Machine

The pressing machine 50 presses the portion forming the upper wall surface 222b of the second accommodation portion 222 of the test container 211, that is, the portion 214A of the container towards the inside of the second accommodation portion 222 by the plunger 52. In this example, the pressing machine 50 further comprises a cylinder 54 which guides the plunger 52 during the pressing operation.

The pressing machine 50 is configured to press a position of the portion 214A of the test container 211 closer to the first flow path 231 from the center in the liquid feeding direction by the plunger 52. By setting the test container 211 on the liquid feeding device 201, the liquid feeding device 201 may be configured so the plunger 52 and the portion 214A of the test container 211 are positioned to have the above relationship, and the plunger 52 and the test container 211 can relatively move so that the plunger 52 of the pressing machine 50 is at a position closer to the first flow path 231 of the portion 214A. For example, the liquid feeding device 1 may include a movement mechanism for the plunger 52 and a movement mechanism of the test container 211.

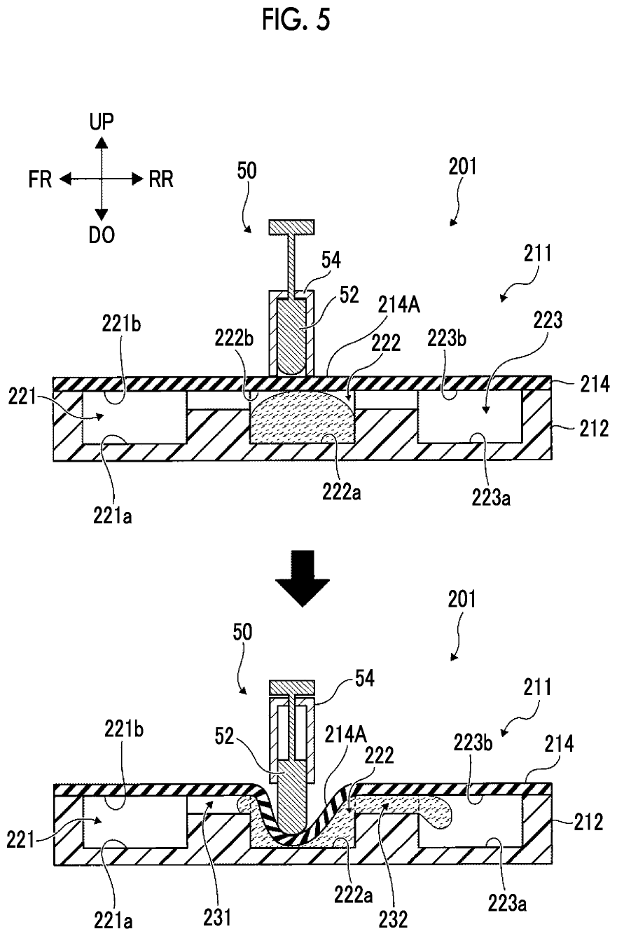

FIG. 5 shows a state before the pressing by the pressing machine 50 (upper diagram) and a state at the time of pressing (lower diagram). As shown in the lower diagram of FIG. 5, the pressing machine 50 presses the portion 214A toward the inside of the second accommodation portion 222 with the plunger 52, thereby deforming the portion 214A forming the upper wall surface 222b of the second accommodation portion 222 to the second accommodation portion 222 side. Accordingly, the volume of the second accommodation portion 222 is reduced and a liquid L in the second accommodation portion 222 is fed to the third accommodation portion 223.

In the liquid feeding device 201, the pressing machine 50 presses the position of the portion 214A of the test container 211 closer to the first flow path 231 with the plunger 52, so that the liquid accommodated in the second accommodation portion 222 moves more to the second flow path 232 side. Accordingly, it is possible to feed a larger amount of liquid to the third accommodation portion 223 and to relatively suppress the amount of a backflow to the first accommodation portion 221 side.

Here, in the plan view shown in FIG. 4, “the position of the portion forming the upper wall surface of the second accommodation portion closer to the first flow path” refers to the position of the portion 214A corresponding to the upper wall surface 222b of the second accommodation portion 222 in a region (shaded region in FIG. 4) closer to the first flow path 231 than a line B1 orthogonal to a center O1 of a licloser to line A1 connecting a boundary C1 with the first flow path 231 of the second accommodation portion 222 and the boundary C2 with the second flow path 232 of the second accommodation portion 222. Here, in a case where the boundary C1 between the first flow path 231 and the second accommodation portion 222 is at 0% position and the boundary C2 between the second accommodation portion 222 and the second flow path 231 is at 100% position, the center O1 is 50% position, and the position closer to the first flow path 231 is a position less than the 50% position. The pressed position is preferably 10% to 30%, and more preferably 10% to 15%.

The pressing portion included in the pressing machine 50 is not limited to the plunger as long as it can press the portion 214A toward the inside of the second accommodation portion 222, and a rod-shaped pressing indenter, a cylinder and the like can be selected. In addition, as for a tip shape, it is possible to appropriately select a shape such as a cylinder, a prism, a hemisphere, a cone, a polygonal pyramid, a flat shape, or a wedge shape.

The portion 214A of the container main body 210 that forms the upper wall surface 222b of the second accommodation portion 222 may be pushed toward the inside of the second accommodation portion 222 to reduce the volume of the second accommodation portion 222 and is not limited to the portion 214A having flexibility throughout. For example, the portion of the portion 214A which directly comes into contact with the plunger 52 may not have flexibility and only the surrounding portion thereof may have flexibility.

In a case where the portion 214A is formed of a flexible film or in a case where the entire upper lid member 214 is formed of a flexible film, a breaking elongation of the flexible film is preferably 100% to 600%, more preferably 200% to 500%, and even more preferably 200% to 400%. In addition, in a case where a thickness of the flexible film is t μm, a modulus of elasticity of the flexible film is α MPa (megapascal), and a depth of the second accommodation portion 22 is d μm, relationships of 0.03≤t/d≤2.5 and 2,000≤α×t≤250,000 are preferably satisfied. The relationships of 0.03≤t/d≤1.8 and 2,000≤α×t≤110,000 are more preferably satisfied, relationships of 0.08≤t/d≤1.0 and 2,000≤α×t≤50,000 are even more preferably satisfied, and relationships of 0.2≤t/d≤0.4 and 4,000≤α×t≤20,000 are particularly preferably satisfied.

As a material of the flexible film, a silicone resin, a fluororesin, polyolefin, polycarbonate, and the like are suitable.

A dispensing port for dispensing a liquid may be provided in a portion of the upper lid member 214 that forms each of the upper wall surfaces 221b, 222b, and 223b of the first accommodation portion 221, the second accommodation portion 222, and the third accommodation portion 223. The dispensing port is opened at the time of dispensing but is preferably sealed at other times. Alternatively, the upper lid member 214 may be provided with no dispensing port, and the upper lid member 214 may be covered and adhered to an upper surface of the main body portion 212 after injecting the liquid to each of the accommodation portions 221, 222, and 223.

In this example, the first flow path 231 and the second flow path 232 have a width narrower than a width of the first accommodation portion 221 and the second accommodation portion 222. The widths of the first flow path 231 and the second flow path 232 may be the same as the widths of the first accommodation portion 221 and the second accommodation portion 222, but smaller width than the widths of the first accommodation portion 221 and the second accommodation portion 222 is more preferable. The width of the flow path 230 is preferably ½ or less and more preferably ⅓ or less of the width of the first accommodation portion 221. The width of the first flow path 231 and the width of the second flow path 232 may or may not be the same.

As the material of the container main body 210, that is, the main body portion 212, any known resin-molded plastic materials can be used without particular limitation. Examples thereof include an acrylic resin such as a polymethyl methacrylate resin (PMMA), a polyolefin resin such as a polycarbonate resin, polyethylene (PE), polypropylene (PP), an ethylene-vinyl acetate copolymer (EVA), a cycloolefin resin such as a cycloolefin polymer (COP) and a cyclic olefin copolymer (COC), a silicone resin, a fluororesin, a polystyrene resin, a polyvinyl chloride resin, a phenol resin, a urethane resin, a polyester resin, an epoxy resin, and a cellulose resin. Particularly, from viewpoints of heat resistance and transparency, a polycarbonate resin, polypropylene, a cycloolefin resin, a silicone resin, and a fluororesin are preferable. In addition, a copolymer of these resins may be used.

A size (volume) of the first accommodation portion 221, the second accommodation portion 222, and the third accommodation portion 223 is, for example, approximately 1 μL (microliter) to several hundreds μL.

Liquid Feeding Device of Second Embodiment

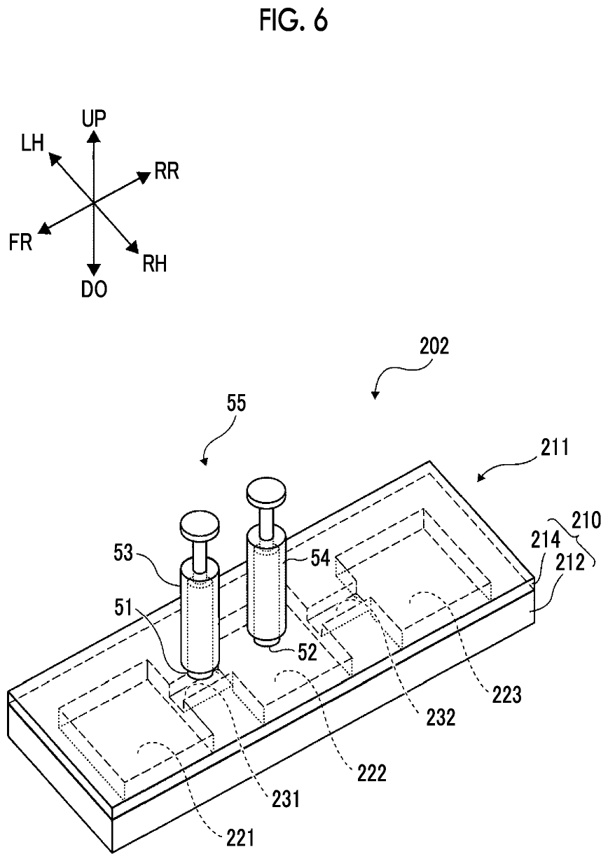

A liquid feeding device of the second embodiment will be described. FIG. 6 is a perspective view showing a schematic configuration of a liquid feeding device 202. The liquid feeding device 202 comprises a test container 211 and a pressing machine 55 having a plunger 51 as the first pressing portion and a plunger 52 as the second pressing portion.

The test container 211 is the same as that of the first embodiment, and is formed of the main body portion 212 and the upper lid member 214 having flexibility over the entire body. However, the test container used in the liquid feeding device 201 of the second embodiment may comprise first accommodation portion 221, a second accommodation portion 222, and a third accommodation portion 223 each accommodating a liquid, a first flow path 231 connecting the first accommodation portion 221 and the second accommodation portion 222 to each other at respective upper end positions thereof, and a second flow path 232 connecting the second accommodation portion 222 and the third accommodation portion 223 to each other at respective upper end positions thereof, in which at least the first portion 214B (see FIG. 2) forming an upper wall surface 231b of the first flow path 231 and a second portion 214A forming an upper wall surface 222b of the second accommodation portion 222 has flexibility.

Pressing Machine

The pressing machine 55 presses the first portion 214B forming the upper wall surface 231b of the first flow path 231 of the test container 211 towards the inside of the first flow path 231 by the first plunger 51, and presses the second portion 214A forming the upper wall surface 222b of the second accommodation portion 222 of the test container 211 towards the inside of the second accommodation portion 222 by the second plunger 52. The pressing machine 50 further comprises a cylinder 53 which guides the plunger 51, and a cylinder 54 which guides the movement of the plunger 52 during the pressing operation. The pressing machine 55 comprises the plungers 51 and 52 so as to press the first portion 214B and the second portion 214A of the test container 211, respectively. By setting the test container 211 on the liquid feeding device 202, the liquid feeding device 202 may be configured so the plunger 51 and the plunger 52 and the first portion 214B and the second portion 214A of the test container 211 are positioned to have the above relationship, and the plunger 51 is provided to relatively move with respect to the test container 211, so that the plunger 51 of the pressing machine 50 is positioned on the first portion 214B, and the plunger 52 is provided to relatively move with respect to the test container 211, so that the plunger 52 is positioned on the first portion 214A. For example, the liquid feeding device 201 may comprise a movement mechanism that moves the plungers 51 and 52 independently or together, a movement mechanism for the test container, or both.

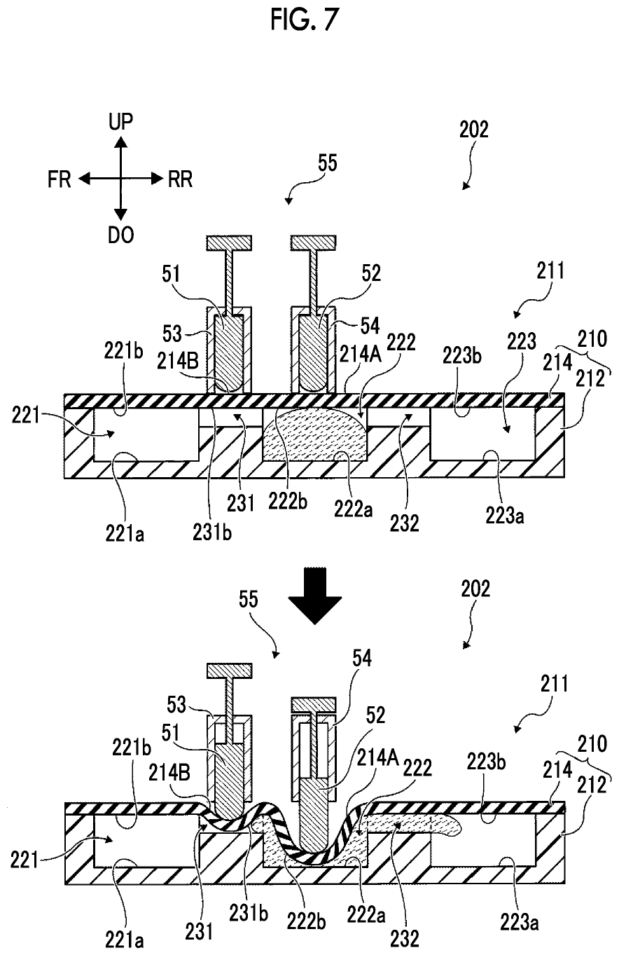

FIG. 7 shows a state before the pressing by the pressing machine 55 (upper diagram) and a state at the time of pressing (lower diagram). As shown in the lower diagram of FIG. 7, the pressing machine 55 presses the portion 214A toward the inside of the second accommodation portion 222 with the plunger 52, thereby deforming the second portion 214A forming the upper wall surface 222b of the second accommodation portion 222 to the second accommodation portion 222 side. Accordingly, the volume of the second accommodation portion 222 is reduced and a liquid L in the second accommodation portion 222 is fed to the third accommodation portion 223. In this case, the pressing machine 55 presses the first portion 214B towards the inside of the first flow path 231 with the plunger 51, the first portion 214B forming the upper wall surface 222b of the second accommodation portion 222 is deformed to the first flow path 231 side, and accordingly, the first flow path 231 is blocked so that the liquid L pushed from the second accommodation portion 222 does not pass through the first flow path 231.

In the liquid feeding device 201, the pressing machine 55 presses the first portion 214B with the first plunger 51 to block the first flow path 231 and presses the second portion 214A with the second plunger 52. Therefore, the liquid accommodated in the second accommodation portion 222 hardly passes through the first flow path 231 and it is possible to effectively prevent the backflow to the first accommodation portion 221, thereby allowing a larger amount of liquid L to flow into the third accommodation portion 223.

The pressing machine 55 may perform the pressing operation by the first plunger 51 and the pressing operation by the second plunger 52 at the same time, or the pressing operation by the first plunger 51 may be performed before the pressing operation by the second plunger 52. The pressing operation by the second plunger 52 may be performed while the pressing state by the first plunger 51 is maintained.

The pressing machine 55 first presses the first flow path 231 with the first plunger 51, and performs a pressing operation with the second plunger 52 while maintaining the state, so that the amount of liquid passing through the first flow path 231 can be further reduced.

In a case of pressing the first portion 214B with the first plunger 51, in the flow path length direction of the first flow path 231, in a case where the boundary C0 with the first accommodation portion 221 is set as the 0% position and the boundary C1 with the second accommodation portion 222 is set as the 100% position, it is preferable to press the second accommodation portion 222 side from the 25% position, more preferable to press the second accommodation portion 222 side from the 50% position, and particularly preferable to press the position of 50% to 75%.

The first pressing portion provided in the pressing machine 55 may be configured to press the first portion 214B toward the inside of the first flow path 231, and is not limited to the plunger. In the same manner, the second pressing portion may be configured to press the second portion 214A toward the inside of the second accommodation portion 222, and is not limited to the plunger.

In the liquid feeding device 201 of the first embodiment and the liquid feeding device 202 of the second embodiment, instead of the test container 211, it is more preferable to comprise the test container comprising the liquid return prevention structure which prevents a backflow of the liquid L to the first accommodation portion 221, in a case where the liquid L accommodated in the second accommodation portion 222 is fed to the third accommodation portion 223 via the second flow path 232. An example of a test container comprising the liquid return prevention structure will be described below.

Test Container 1

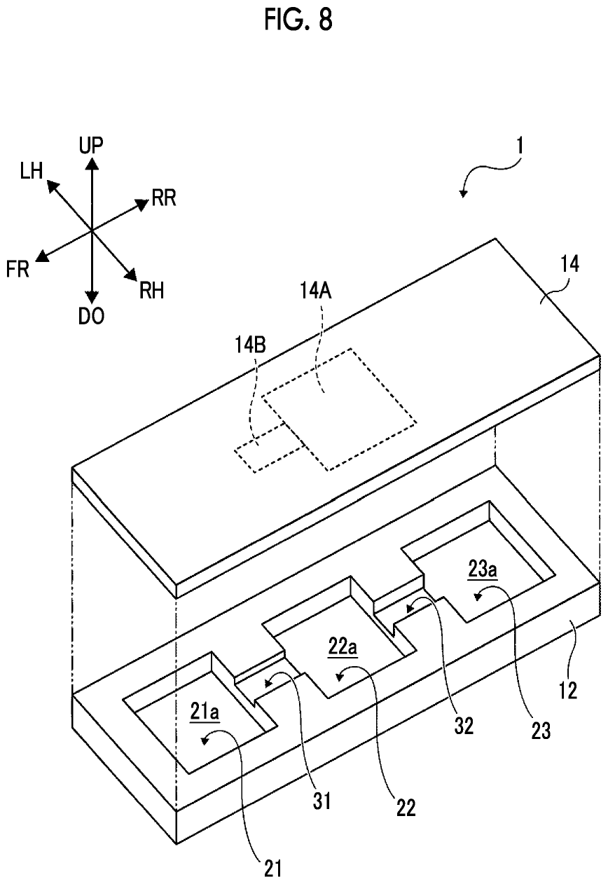

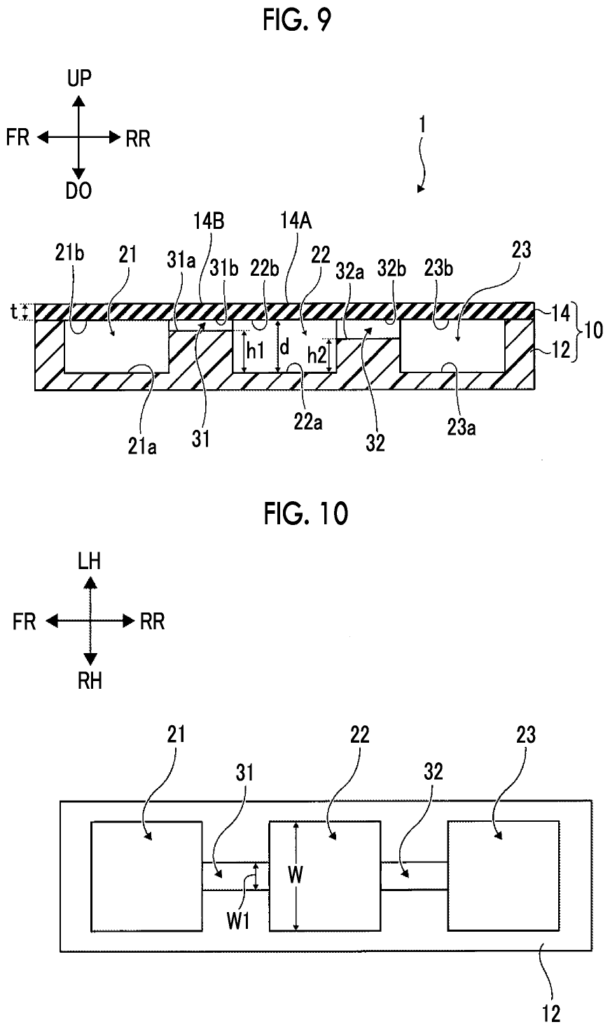

The test container 1 will be described. FIG. 8 is an exploded perspective view showing a schematic configuration of a test container 1. FIG. 9 is an cross-sectional view showing a schematic configuration of the test container 1. FIG. 10 is a plan view showing a schematic configuration of a main body portion 12 of the test container 1.

The test container 1 shown in FIGS. 8, 9, and 10 comprises the container main body 10 including a first accommodation portion 21, a second accommodation portion 22, and a third accommodation portion 23 each accommodating a liquid, a first flow path 31 connecting the first accommodation portion 21 and the second accommodation portion 22 to each other at respective upper end positions thereof, and a second flow path 32 connecting the second accommodation portion 22 and the third accommodation portion 23 to each other at respective upper end positions thereof. The container main body 10 has at least a portion 14A forming an upper wall surface 22b of the second accommodation portion 22 having flexibility to be deformable inwards of the second accommodation portion 22. In a case of applying to the liquid feeding device 202, the portion 14B forming the upper wall surface 31b of the first flow path 31 further has flexibility deformable toward the inside of the first flow path 31.

In this example, the container main body 10 comprises a main body portion 12 and an upper lid member 14. The main body portion 12 has an opening in a portion forming each of the first accommodation portion 21, the first flow path 31, the second accommodation portion 22, the second flow path 32, and the third accommodation portion 23. The container main body 10 has a configuration in which the first accommodation portion 21, the first flow path 31, the second accommodation portion 22, the second flow path 32, and the third accommodation portion 23 are formed therein by covering the opening of the main body portion 12 with the upper lid member 14. In other words, the main body portion 12 configures the inner bottom surfaces 21a to 23a and the side wall surfaces of the accommodation portions 21 to 23, and the inner bottom surfaces 31a and 32a and the side wall surfaces of the flow paths 31 and 32, and the upper lid member 14 configures the upper wall surfaces 21b to 23b of the accommodation portions 21 to 23 and the upper wall surfaces 31b and 32b of the flow paths 31 and 32. However, the present invention is not limited to this configuration, as long as it has a configuration of comprising each accommodation portion and each flow path therein.

In this example, the upper lid member 14 has flexibility throughout. However, the entire upper lid member 14 does not have to be flexible, as long as the portion 14A configuring at least the upper wall surface 22b of the second accommodation portion 22 of the container main body 10, that is, the portion 14A of the upper lid member 14 has a flexible portion deformable in a direction toward the second accommodation portion 22.

As a liquid return prevention structure, the test container 1 has a structure in which a height h1 from the inner bottom surface 22a of the second accommodation portion 22 to the inner bottom surface 31a of the first flow path 31 (hereinafter, referred to as a “height h1 of the first flow path”) is higher than a height h2 from the inner bottom surface 22a of the second accommodation portion 22 to the inner bottom surface 32a of the second flow path 32 (hereinafter, referred to as a “height h2 of the second flow path”). In the test container 1, the height h1 of the inner bottom surface 31a of the first flow path 31 from the inner bottom surface 22a of the second accommodation portion 22 is defined as a height of a corner of a level difference portion between the first flow path 31 and the second accommodation portion 22 from the inner bottom surface 22a of the second accommodation portion 22. In the same manner, the height h2 of the inner bottom surface 32a of the second flow path 32 from the inner bottom surface 22a of the second accommodation portion 22 is defined as a height of a corner of a level difference portion between the second accommodation portion 22 and the second flow path 32 from the inner bottom surface 22a of the second accommodation portion 22. The liquid return prevention structure is a structure for preventing a backflow of the liquid to the first accommodation portion 21, in a case where the liquid accommodated in the second accommodation portion 22 is fed to the third accommodation portion 23 via the second flow path 32 due to the deformation of the portion 14A forming the upper wall surface 22b of the second accommodation portion 22 in a direction toward the second accommodation portion 22.

The test container 1 comprises the first flow path 31 at the upper end position of the first accommodation portion 21 and the second accommodation portion 22, and the second flow path 32 at the upper end position of the second accommodation portion 22 and the third accommodation portion 23, respectively. Accordingly, the liquid accommodated in the accommodation portion is difficult to flow into the flow path, compared to a case where the flow path is comprised at a lower end or in the middle in a depth direction. Therefore, it is possible to prevent a passage of the liquid into the flow path due to a capillary phenomenon or the like without applying an external force. Meanwhile, since the portion 14A deformable toward the inside of the second accommodation portion 22 is comprised at the upper portion of the second accommodation portion 22, the portion 14A is deformed toward the inside of the second accommodation portion 22 to reduce a volume of the second accommodation portion 22, thereby easily realizing liquid feeding to the third accommodation portion 23 by pushing the liquid accommodated in the second accommodation portion 22.

Since the height h1 of the first flow path 31 is higher than the height h2 of the second flow path 32, in a case where the portion 14A of the container main body 10 is deformed in the direction toward the second accommodation portion 22 so that the liquid accommodated in the second accommodation portion 22 is fed to the third accommodation portion 23 via the second flow path 32, the liquid pushed from the second accommodation portion 22 is preferentially fed to the second flow path 32 formed at a lower position. Accordingly, the liquid return to the first flow path 31 can be suppressed, and the liquid feeding properties to the third accommodation portion 23 at a downstream side is high. According to this configuration, it is possible to suppress the liquid return to the first flow path 31 and increase the liquid feeding properties to the third accommodation portion 23 with a simple configuration of providing a difference between the heights h1 and h2.

A difference h1−h2 between the height h1 of the first flow path 31 and the height h2 of the second flow path 32 is preferably 20% or more, more preferably 30% or more, and even more preferably 50% or more of the height h2 of the second flow path 32. As the difference h1−h2 is large, the liquid feeding to the second flow path 32 is further promoted, and the liquid feeding properties to the third accommodation portion 23 can be increased.

Test Container 1A

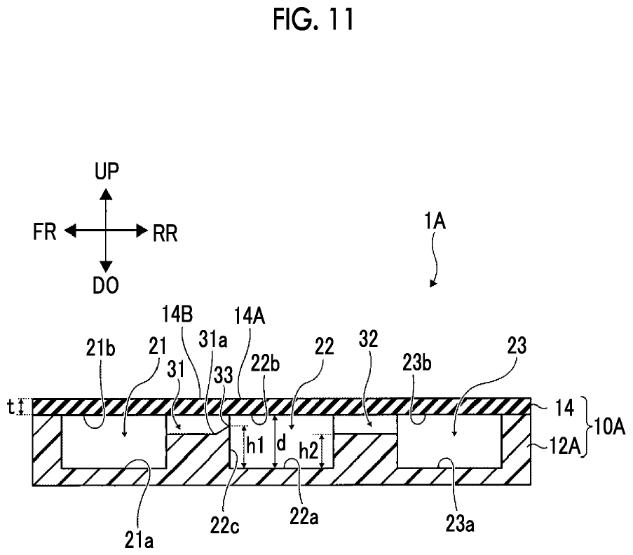

FIG. 11 shows a test container 1A which is a modification example of the test container 1. The test container 1A comprises a container main body 10A consisting of a main body portion 12A and an upper lid member 14. In the test container 1A, a corner 33 formed by an inner bottom surface 31a of the first flow path 31 and an inner side surface 22c of the second accommodation portion 22 in a level difference portion between the inner bottom surface 31a of the first flow path 31 and the second accommodation portion 22 has an acute angle. By setting the corner 33 of the level difference portion to have an acute angle, it is possible to more effectively suppress the flow of the liquid accommodated in the second accommodation portion 22 to the first flow path, compared to a case where the angle is equal to or greater than 90°. Therefore, it is possible to more preferentially feed the liquid accommodated in the second accommodation portion 22 to the second flow path 32.

Test Container 2

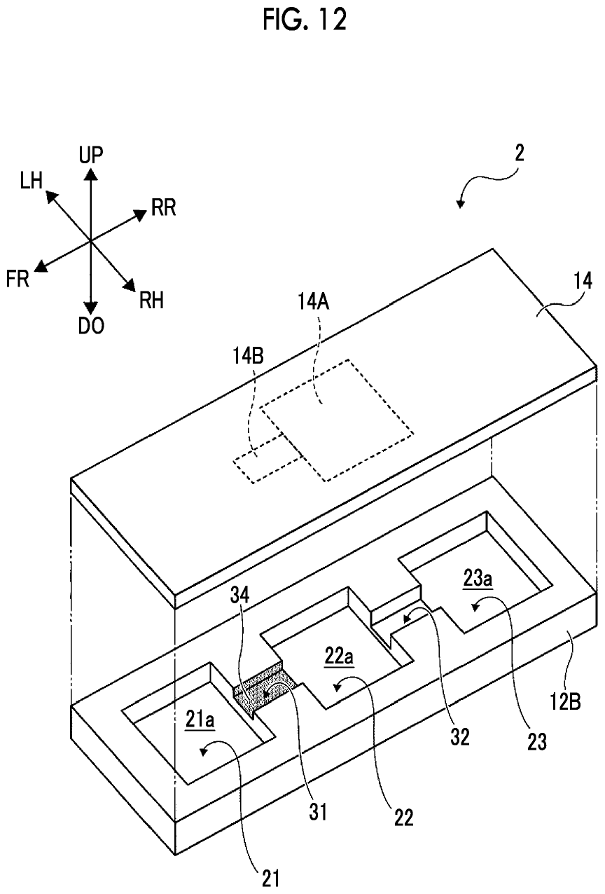

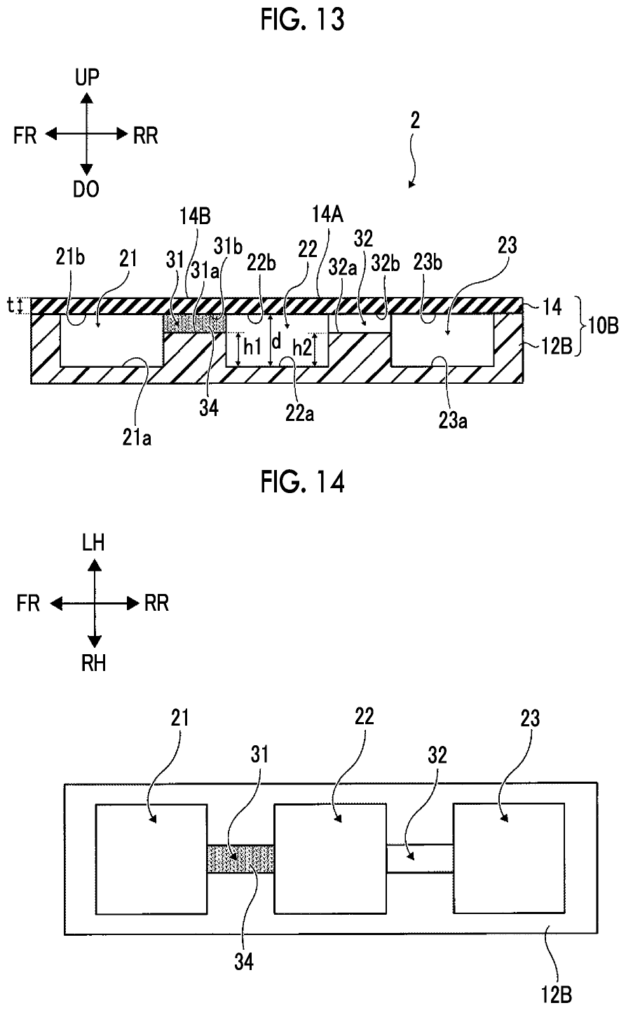

The test container 2 will be described. FIG. 12 is an exploded perspective view showing a schematic configuration of a test container 2. FIG. 13 is an cross-sectional view showing a schematic configuration of the test container 2. FIG. 14 is a plan view showing a schematic configuration of a main body portion 12B of the test container 2.

The test container 2 shown in FIGS. 12, 13, and 14 comprises the container main body 10B including a first accommodation portion 21, a second accommodation portion 22, and a third accommodation portion 23 each accommodating a liquid, a first flow path 31 connecting the first accommodation portion 21 and the second accommodation portion 22 to each other at respective upper end positions thereof, and a second flow path 32 connecting the second accommodation portion 22 and the third accommodation portion 23 to each other at respective upper end positions thereof. The container main body 10B has at least the portion 14A forming the upper wall surface 22b of the second accommodation portion 22 having flexibility to be deformable inwards of the second accommodation portion 22. In a case of applying to the liquid feeding device 202, the portion 14B forming the upper wall surface 31b of the first flow path 31 further has flexibility deformable toward the inside of the first flow path 31. In the drawings, the same reference numerals are used for the same elements as those of the test container 1. Elements having the same reference numerals as those of the test container 1 are the same as those described for the test container 1, and specific description thereof will be omitted. The same applies to the following drawings.

In this example, the container main body 10B comprises the main body portion 12B and the upper lid member 14. The main body portion 12B has an opening in a portion forming each of the first accommodation portion 21, the first flow path 31, the second accommodation portion 22, the second flow path 32, and the third accommodation portion 23. The container main body 10B has a configuration in which the first accommodation portion 21, the first flow path 31, the second accommodation portion 22, the second flow path 32, and the third accommodation portion 23 are formed therein by covering the opening of the main body portion 12B with the upper lid member 14. In other words, the main body portion 12B configures the inner bottom surfaces 21a to 23a and the side wall surfaces of the accommodation portions 21 to 23, and the inner bottom surfaces 31a and 32a and the side wall surfaces of the flow paths 31 and 32, and the upper lid member 14 configures the upper wall surfaces 21b to 23b of the accommodation portions 21 to 23 and the upper wall surfaces 31b and 32b of the flow paths 31 and 32. However, the present invention is not limited to this configuration, as long as it has a configuration of comprising each accommodation portion and each flow path therein.

The test container 2 has a structure of the first flow path 31 and the second flow path 32 in which a water contact angle R1 of the inner surface of the first flow path 31 is set to be greater than a water contact angle R2 of the inner surface of the second flow path 32, as the liquid return prevention structure. In this example, a hydrophobic surface 34 obtained by performing a hydrophobic treatment is formed on the inner surface of the first flow path 31.

In order to generate a difference in a water contact angle between the inner surface of the first flow path 31 and the inner surface of the second flow path 32, the hydrophobic treatment may be performed on the inner surface of the first flow path 31 as in this example and/or a hydrophilic treatment may be performed on the inner surface of the second flow path 32.

The test container 2 comprises the first flow path 31 at the upper end position of the first accommodation portion 21 and the second accommodation portion 22, and the second flow path 32 at the upper end position of the second accommodation portion 22 and the third accommodation portion 23, respectively. Accordingly, the liquid accommodated in the accommodation portion is difficult to flow into the flow path, compared to a case where the flow path is comprised at a lower end or in the middle in a depth direction. Therefore, it is possible to prevent a passage of the liquid into the flow path due to a capillary phenomenon or the like without applying an external force. Meanwhile, since the portion 14A deformable toward the inside of the second accommodation portion 22 is comprised at the upper portion of the second accommodation portion 22, the portion 14A is deformed toward the inside of the second accommodation portion 22 to reduce a volume of the second accommodation portion 22, thereby easily realizing liquid feeding to the third accommodation portion 23 by pushing the liquid accommodated in the second accommodation portion 22.

The portion 14A of the container main body 10B is deformed in the direction toward the second accommodation portion 22, so that the liquid accommodated in the second accommodation portion 22 is fed to the third accommodation portion 23 via the second flow path 32. In this case, since the water contact angle of the inner surface of the first flow path 31 is greater than the water contact angle of the inner surface of the second flow path 32, the liquid pushed from the second accommodation portion 22 is preferentially fed to the second flow path 32 having a smaller water contact angle. Accordingly, the liquid return to the first flow path 31 can be suppressed, and the liquid feeding properties to the third accommodation portion 23 at a downstream side is high. According to this configuration, it is possible to suppress the liquid return to the first flow path 31 and increase the liquid feeding properties to the third accommodation portion 23 with a simple process of only the surface treatment.

The surface treatment such as the hydrophilic treatment or the hydrophobic treatment is preferably formed on the entire inner surface of each flow path, but a part of the inner surface may not be treated.

Examples of the hydrophilic treatment include a surface modification treatment such as a corona treatment, a plasma treatment, an ozone treatment, a treatment of applying a hydrophilic coating agent, and bonding of a hydrophilic film. Examples of the hydrophobic treatment include a treatment of applying a hydrophobic coating agent such as a fluororesin or a hydrophobic silica-containing resin, a silane coupling treatment, and bonding of a water-repellent film.

A difference R1−R2 between the water contact angle R1 of the first flow path 31 and the water contact angle R2 of the second flow path 32 is preferably 10° or more, more preferably 20° or more, even more preferably 40° or more, and further preferably 60° or more.

In the present specification, the water contact angle is a contact angle of pure water. Specifically, 1 μL of pure water is added dropwise to the inner surface of the flow path and the accommodation portion under the condition of an atmosphere temperature of 25° C., the contact angle is measured by the θ/2 method using a fully-automatic contact angle meter (model number: DM-701, Kyowa Interface Science Co., Ltd.), and an arithmetic mean value of values obtained by measuring 5 times is used.

Test Container 3

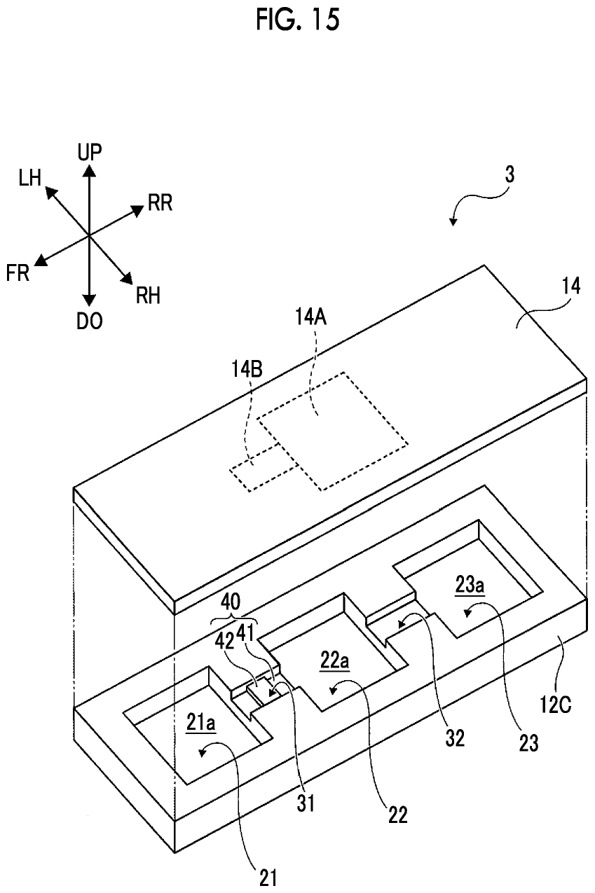

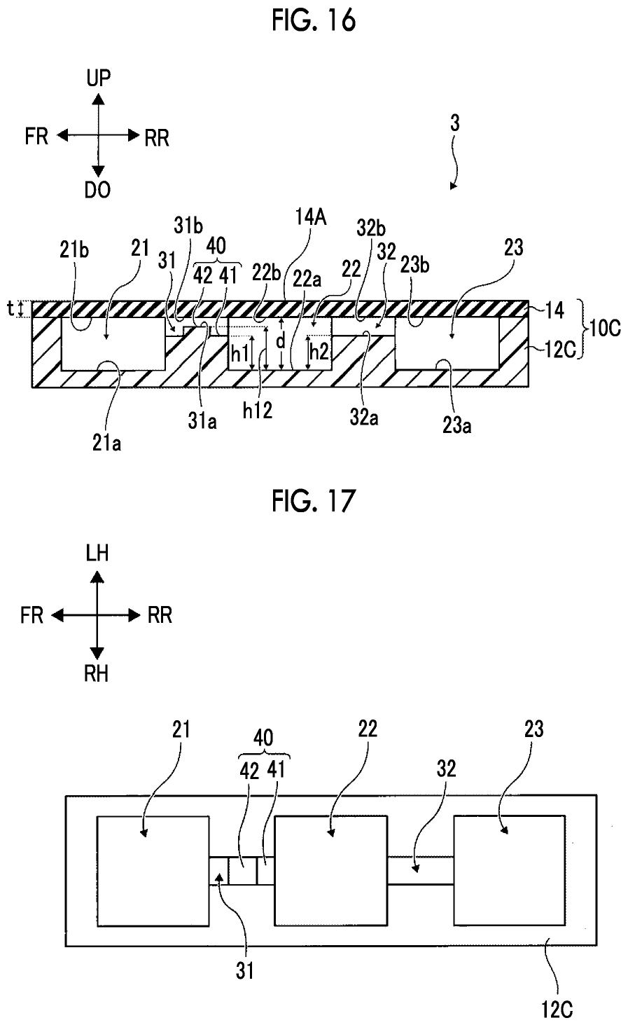

The test container 3 will be described. FIG. 15 is an exploded perspective view showing a schematic configuration of a test container 3. FIG. 16 is an cross-sectional view showing a schematic configuration of the test container 3. FIG. 17 is a plan view showing a schematic configuration of a main body portion 12C of the test container 3.

The test container 3 shown in FIGS. 15, 16, and 17 comprises the container main body 10C including a first accommodation portion 21, a second accommodation portion 22, and a third accommodation portion 23 each accommodating a liquid, a first flow path 31 connecting the first accommodation portion 21 and the second accommodation portion 22 to each other at respective upper end positions thereof, and a second flow path 32 connecting the second accommodation portion 22 and the third accommodation portion 23 to each other at respective upper end positions thereof. The container main body 10C has at least the portion 14A forming the upper wall surface 22b of the second accommodation portion 22 having flexibility to be deformable inwards of the second accommodation portion 22. In a case of applying to the liquid feeding device 202, the portion 14B forming the upper wall surface 31b of the first flow path 31 further has flexibility deformable toward the inside of the first flow path 31.

In this example, the container main body 10C comprises the main body portion 12C and the upper lid member 14. The main body portion 12C has an opening in a portion forming each of the first accommodation portion 21, the first flow path 31, the second accommodation portion 22, the second flow path 32, and the third accommodation portion 23. The container main body 10C has a configuration in which the first accommodation portion 21, the first flow path 31, the second accommodation portion 22, the second flow path 32, and the third accommodation portion 23 are formed therein by covering the opening of the main body portion 12C with the upper lid member 14. That is, the main body portion 12C constitutes the inner bottom surfaces 21a to 23a and the side wall surfaces of the accommodation portions 21 to 23, and the inner bottom surfaces 31a and 32a and the side wall surfaces of the flow paths 31 and 32, respectively. The upper lid member 14 configures the upper wall surfaces 21b to 23b of the accommodation portions 21 to 23 and the upper wall surfaces 31b and 32b of the flow paths 31 and 32. However, the present invention is not limited to this configuration, as long as it has a configuration of comprising each accommodation portion and each flow path therein.

The test container 3 has a structure of a stepped portion 40 which is provided on the second accommodation portion 22 side of the first flow path 31 and which includes two or more steps 41 and 42 from the inner bottom surface 22a of the second accommodation portion 22, as the liquid return prevention structure. On the other hand, the second flow path 32 does not comprise a stepped portion. In addition, in this example, the stepped portion is provided on the first accommodation portion 21 side of the first flow path 31, but the stepped portion may not be provided on the first accommodation portion 21 side.

The test container 3 comprises the first flow path 31 at the upper end position of the first accommodation portion 21 and the second accommodation portion 22, and the second flow path 32 at the upper end position of the second accommodation portion 22 and the third accommodation portion 23, respectively. Accordingly, the liquid accommodated in the accommodation portion is difficult to flow into the flow path, compared to a case where the flow path is comprised at a lower end or in the middle in a depth direction. Therefore, it is possible to prevent a passage of the liquid into the flow path due to a capillary phenomenon or the like without applying an external force. Meanwhile, since the portion 14A deformable toward the inside of the second accommodation portion 22 is comprised at the upper portion of the second accommodation portion 22, the portion 14A is deformed toward the inside of the second accommodation portion 22 to reduce a volume of the second accommodation portion 22, thereby easily realizing liquid feeding to the third accommodation portion 23 by pushing the liquid accommodated in the second accommodation portion 22.

The portion 14A of the container main body 10C is deformed in the direction toward the second accommodation portion 22, so that the liquid accommodated in the second accommodation portion 22 is fed to the third accommodation portion 23 via the second flow path 32. In this case, since the first flow path 31 comprises the stepped portion 40 having two or more steps, a barrier in a case where the liquid accommodated in the second accommodation portion 22 passes through the first flow path 31 has two or more steps. Accordingly, the invasion of the liquid into the first flow path 31 is suppressed, and the liquid pushed out from the second accommodation portion 22 is preferentially fed to the second flow path 32 having a smaller barrier. Therefore, the liquid return to the first flow path 31 is suppressed, and the liquid feeding properties to the third accommodation portion 23 at a downstream side is high. It is possible to obtain a high effect of preventing the liquid return to the first flow path 31 by providing the stepped portion 40 in the first flow path 31.

The stepped portion 40 includes a first step 41 on the second accommodation portion 22 side and a second step 42. The stepped portion 40 is not limited to two steps and may have three steps or four or more steps. However, from a viewpoint of avoiding complication of the structure, the stepped portion 40 preferably has two or three steps.

The height h1 of the first step 41 is preferably 25% or more, more preferably 30% or more, and even more preferably 50% or more of d, where d is a height (depth) from the inner bottom surface 22a to the upper wall surface 22b of the second accommodation portion 22.

A height h12 of the second step 42 is preferably 50% or more, more preferably 60% or more, and even more preferably 80% or more of the height d of the second accommodation portion 22. A difference between the height h12 of the second step 42 and the height h1 of the first step 41 is preferably 20% or more of the height h1 of the first step 41, from a viewpoint of preventing the liquid return. The height h12 of the second step 42 is defined as a height from the inner bottom surface 22a of the second accommodation portion 22 at the corner of the level difference portion with the first step 41.

Test Container 3A

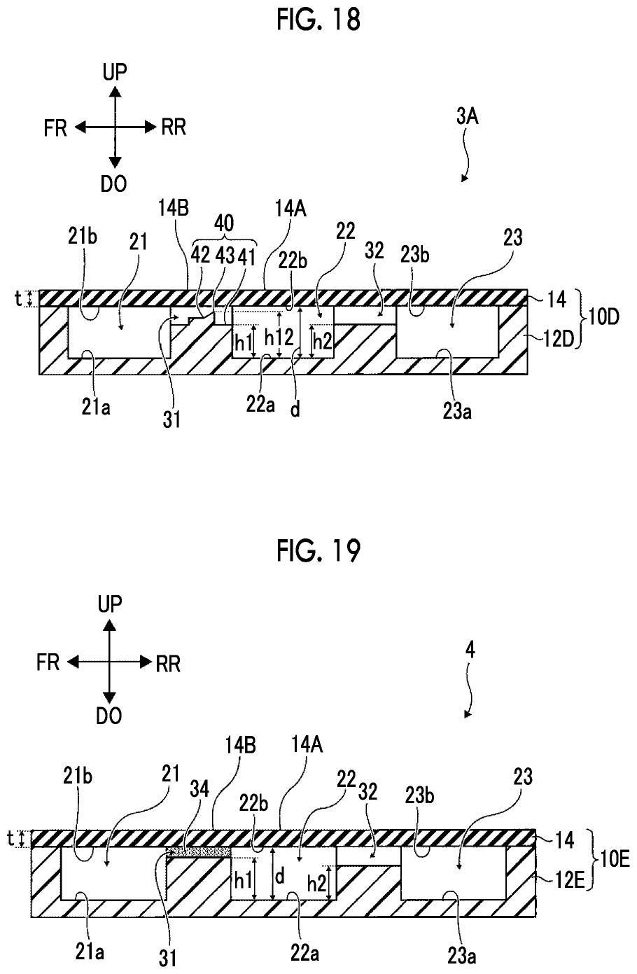

FIG. 18 shows a test container 3A of a modification example of the third embodiment. The test container 3A comprises a container main body 10D consisting of a main body portion 12D and the upper lid member 14. In the test container 3A, a corner (here, a corner 43) formed by the inner bottom surface and the inner side surface forming at least one step of the stepped portion 40 has an acute angle. By setting the corner 43 of the level difference portion to have an acute angle, it is possible to more effectively suppress the flow of the liquid accommodated in the second accommodation portion 22 to the first flow path 31, compared to a case where the angle is equal to or greater than 90°. Therefore, it is possible to more preferentially feed the liquid accommodated in the second accommodation portion 22 to the second flow path 32.

In the test container 3A shown in FIG. 18, only the corner 43 of the second step 42 of the stepped portion 40 has an acute angle, but corners of all of the steps included in the flow path 31 may have an acute angle. In a case where at least one corner of the steps 41 and 42 of the stepped portion 40 is an acute angle, the effect of suppressing the flow of the liquid from the second accommodation portion 22 into the first flow path 31 is improved.

The liquid feeding method of the liquid in the present test container 3 or the modification example thereof is the same as that in the case of the test container 1 of the first embodiment.

As described above, the test container 1 comprises a structure in which the height h1 of the first flow path 31 is higher than the height h2 of the second flow path 32 (hereinafter, referred to as a liquid return prevention structure 1). The test container 2 comprises a structure of the first flow path 31 and the second flow path 32 in which the water contact angle of the inner surface of the first flow path 31 is set to be greater than the water contact angle of the inner surface of the second flow path 32 (hereinafter, referred to as a liquid return prevention structure 2). The test container 3 has a structure of the stepped portion 40 including two or more steps from the inner bottom surface 22a of the second accommodation portion 22 configured on the second accommodation portion side of the first flow path 31 (hereinafter, referred to as a liquid return prevention structure 3).

It is also preferable to comprise these liquid return prevention structures 1 to 3 in combination. For example, as shown in FIG. 19, a test container 4 comprising the liquid return prevention structure 1 and the liquid return prevention structure 2 may be used. The test container 4 comprises a container main body 10E formed of a main body portion 12E and the upper lid member 14. The test container 4 has a structure in which the height h1 of the first flow path and the height h2 of the second flow path satisfy a relationship of h1>h2 and comprises the hydrophobic surface 34 obtained by performing a hydrophobic treatment on the inner surface of the first flow path 31, and the water contact angle of the inner surface of the first flow path 31 is higher than the water contact angle of the inner surface of the second flow path 32.

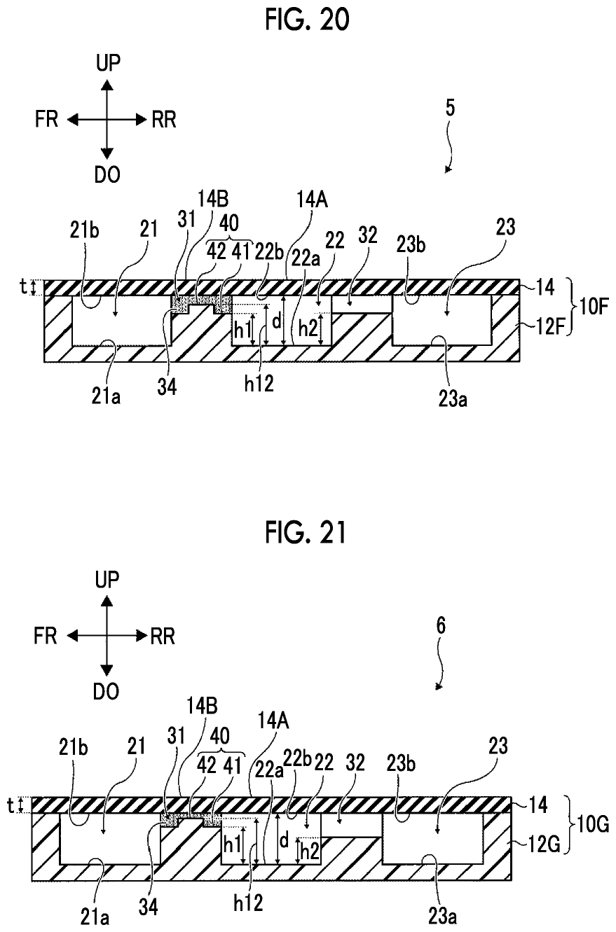

For example, as shown in FIG. 20, a test container 5 comprising the liquid return prevention structure 2 and the liquid return prevention structure 3 may be used. The test container 5 comprises a container main body 10F forming of a main body portion 12F and the upper lid member 14. The test container 5 comprises the hydrophobic surface 34 obtained by performing a hydrophobic treatment on the inner surface of the first flow path 31 and comprises the stepped portion 40 in the first flow path 31, and the water contact angle of the inner surface of the first flow path 31 is higher than the water contact angle of the inner surface of the second flow path 32.

In addition, a test container comprising the liquid return prevention structure 1 and the liquid return prevention structure 3 may be used, or, as shown in FIG. 21, a test container 6 comprising all of the liquid return prevention structures 1 to 3 may be used. The test container 6 comprises a container main body 10G formed of a main body portion 12G and the upper lid member 14. The test container 6 has a structure in which the height h1 of the first flow path 31 and the height h2 of the second flow path 32 satisfy a relationship of h1>h2 and comprises the hydrophobic surface 34 obtained by performing a hydrophobic treatment on the inner surface of the first flow path 31, and the water contact angle of the inner surface of the first flow path 31 is higher than the water contact angle of the inner surface of the second flow path 32. In addition, the first flow path 31 comprises the stepped portion 40.

According to the test container comprising two or three the liquid return prevention structures 1 to 3 in combination, it is possible to obtain a higher effect of the liquid return prevention, compared to a case of comprising only the liquid return prevention structure 1, only the liquid return prevention structure 2, or only the liquid return prevention structure 3.

In addition, in the test container of the present disclosure, the liquid return prevention structure is not limited to the above example, and the first flow path between the second accommodation portion and the first accommodation portion may have a structure in which the liquid accommodated in the second accommodation portion relatively hardly flows, compared to the second flow path between the second accommodation portion and the third accommodation portion. For example, a structure including a valve may be comprised in each of the first flow path and the second flow path may be provided as the liquid return prevention structure. In a case where a valve is provided in each of the first flow path and the second flow path, the liquid is fed in a state where the valve of the first flow path is closed and valve of the second flow path is opened, in a case of feeding the liquid from the second accommodation portion to the third accommodation portion, it is possible to effectively prevent the liquid return to the first accommodation portion and improve the liquid feeding properties to the third accommodation portion.

Nucleic Acid Extraction Test Device

The liquid feeding device of the technology of the present disclosure can be applied to, for example, a nucleic acid extraction test device. A nucleic acid extraction test device and a nucleic acid extraction test as an embodiment of the liquid feeding device of the present disclosure will be described.

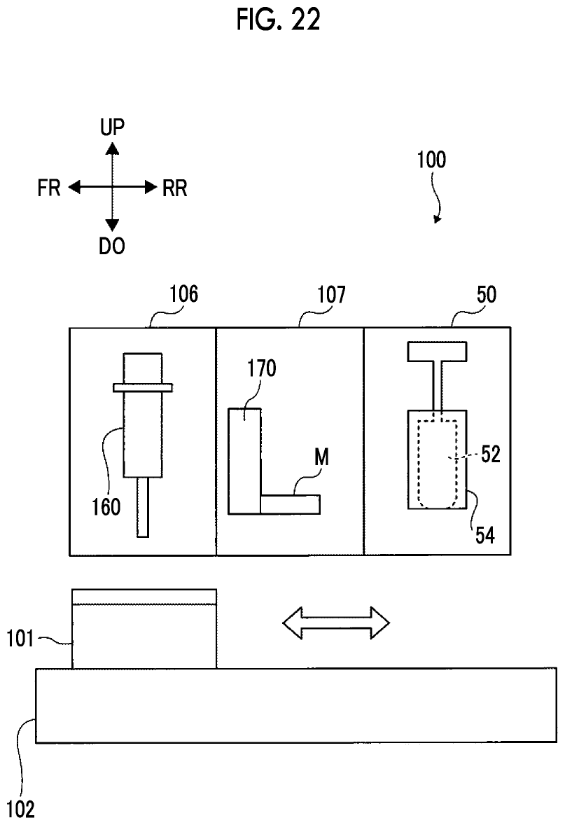

FIG. 22 is a configuration diagram showing a schematic configuration of a nucleic acid extraction test device 100 comprising the test container 101. The nucleic acid extraction test device 100 comprises the test container 101, the pressing machine 50, a dispenser 106, a magnetic field generation and movement unit 107, and a transportation portion 102 for the test container 101.

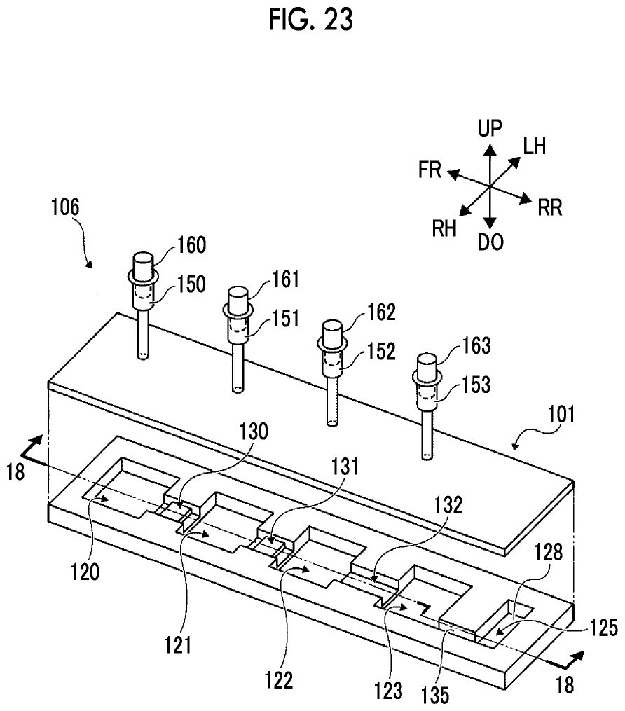

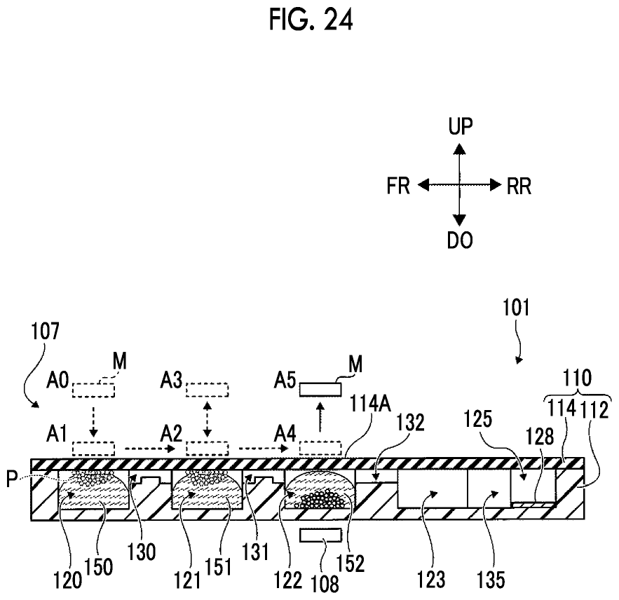

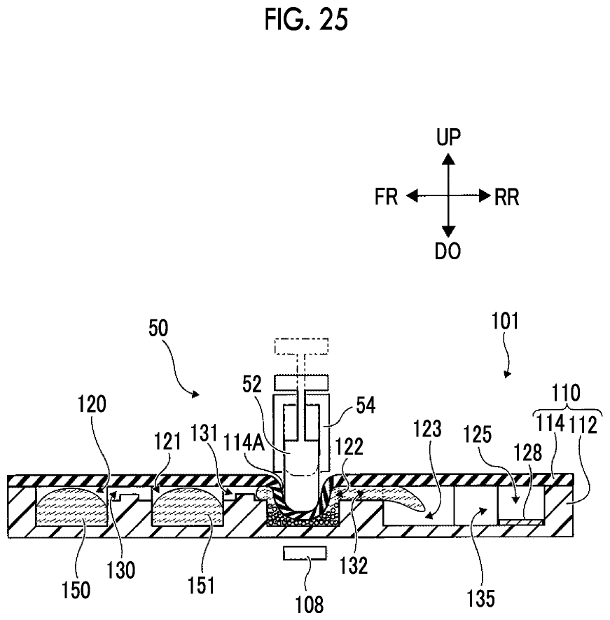

FIG. 23 is an exploded perspective view of a test container 101 and a diagram showing a main part of a dispenser 106. FIG. 24 is a diagram showing the test container 101 and a magnet M of the magnetic field generation and movement unit 107. FIG. 25 is a diagram showing the test container 101 and a main part of the pressing machine 50. FIGS. 24 and 25 show cross-sectional views taken along a line 18-18 of the test container 101 shown in FIG. 23.

The test container 101 comprises a container main body 110 comprising four accommodation portions 120 to 123 accommodating a liquid, respectively, a chromatographic carrier accommodation portion 125 accommodating a chromatographic carrier 128, and four flow paths 130, 131, 132, and 135 therein.

The container main body 110 comprises a main body portion 112 and an upper lid member 114. The main body portion 112 has an opening in a portion forming each of the accommodation portions 120 to 123 and 125 and the flow paths 130, 131, 132, and 135. The container main body 110 has a configuration in which the accommodation portions 120 to 123 and 125 and the flow paths 130, 131, 132, and 135 are formed therein by covering the main body portion 112 with the upper lid member 114. The main body portion 112 configures the side wall surface and the bottom surface of each of the accommodation portions and the flow paths, and the upper lid member 114 configures the upper wall surface of each of the accommodation portions and the flow paths. In this example, the upper lid member 114 is formed of a flexible film. The upper lid member 114 is provided with an injection port (not shown) for injecting the liquid accommodated in each of the accommodation portions 120 to 123. The tips of syringes 160 to 163 are inserted into the injection ports, respectively, and various liquids can be injected into the corresponding accommodation portions 120 to 123.

The accommodation portion 120 is a magnetic particle collecting chamber (hereinafter, referred to as the magnetism collecting chamber 120) which accommodates a specimen solution 150 containing magnetic particles P to which a nucleic acid is adsorbed. The accommodation portion 121 is a cleaning chamber (hereinafter, referred to as a cleaning chamber 121) which accommodates a cleaning solution 151 and cleans a substance non-specifically adsorbed to the magnetic particles P. The accommodation portion 122 is a PCR chamber (hereinafter, referred to as a PCR chamber 122) which accommodates a polymerase chain reaction (PCR) solution 152. The accommodation portion 123 is a detection chamber (hereinafter, referred to as a detection chamber 123) for mixing an amplified nucleic acid and a development solution 153.

The flow path 130 connects a magnetism collecting chamber 120 and the cleaning chamber 121 to each other at an upper end position. The flow path 130 comprises a stepped portion on the sides of the magnetism collecting chamber 120 and the cleaning chamber 121, to suppress the flow of the specimen solution 150 accommodated in the magnetism collecting chamber 120 to the flow path 130 and to prevent the mixing of the specimen solution 150 with the cleaning solution 151 accommodated in the cleaning chamber 121.

The flow path 131 connects the cleaning chamber 121 and the PCR chamber 122 to each other at an upper end position and the flow path 132 connects the PCR chamber 122 and the detection chamber 123 to each other at an upper end position. The cleaning chamber 121, the PCR chamber 122, the detection chamber 123, and the flow paths 131 and 132 correspond to the first accommodation portion, the second accommodation portion, the third accommodation portion, the first flow path, and the second flow path in the technology of the present disclosure, respectively. In addition, here, the liquid return prevention structure of suppressing the backflow of the liquid to the cleaning chamber 121, in a case of feeding the liquid accommodated in the PCR chamber 122 to the detection chamber 123 through the flow path 132 may be comprised. In this example, the liquid return prevention structure 3 is included as the liquid return prevention structure. That is, as the liquid return prevention structure, a structure of a stepped portion including two or more steps from an inner bottom surface 122a of the PCR chamber 122, which is formed on the PCR chamber 122 side of the flow path 131, is comprised.

The liquid return prevention structure may include a structure (liquid return prevention structure 1) in which a height of the first flow path (flow path 131) is higher than a height of the second flow path (flow path 132). In addition, a structure of the first flow path and the second flow path in which the water contact angle of the inner surface of the first flow path is set to be greater than the water contact angle of the inner surface of the second flow path (liquid return prevention structure 2) may be included. Alternatively, two or more of other liquid return prevention structures and liquid return prevention structures 1 to 3 may be provided in combination.

The flow path 132 connects the PCR chamber 122 and the detection chamber 123 at the upper end position. The flow path 132 may comprise a valve (not shown), in order to prevent evaporation of the liquid in a case of adjusting a temperature of the PCR chamber. The valve may be any valve that can be opened in a case where liquid is fed from the PCR chamber 122 to the detection chamber 123.

The flow path 135 connects the detection chamber 123 and the chromatographic carrier accommodation portion 125 to each other at a lower end position.

The magnetic particles P are particles that are attracted by magnetic force. The magnetic particles P are, for example, magnetic particles processed so as to adsorb a specific sample such as DNA. Specifically, as the magnetic particles P, model number: Magnosphere MX100/Carboxyl and model number: Magnosphere MS160/Tosyl manufactured by JSR Corporation, sicastar manufactured by Corefront, Magrapid manufactured by Sanyo Chemical Industries, Ltd. can be used.

As the magnetic particles P, magnetic particles having a particle size in a range of 0.01 μm to 100 μm are used. As the magnetic particles P, magnetic particles having a particle size of approximately 1 μm to 10 μm are preferably used. The magnetic particles P may be comprised in the magnetism collecting chamber 120 in advance, or may be injected into the magnetism collecting chamber 120 together with the specimen solution 150.

The specimen solution 150 is, for example, a specimen solution containing a nucleic acid extracted from a specimen. The specimen solution 150 may include a surfactant for extracting a nucleic acid such as deoxyribonucleic acid (DNA) and ribonucleic acid (RNA) from the specimen and adsorbing the nucleic acid on the surfaces of the magnetic particles P. In addition, as the surfactant, for example, sodium dodecyl sulfate, polyoxyethylene sorbitan monolaurate (Tween 20), Triton X-100, or the like can be used. These surfactants may be used alone or in combination of a plurality thereof. A chaotropic substance such as guanidine hydrochloride may be included in order to promote extraction of nucleic acid from the specimen and surface adsorption to the magnetic particles P. In addition, instead of containing the surfactant, a nucleic acid extracted from a specimen using a column may be contained. In addition, a surfactant for suppressing aggregation of the magnetic particles P may be included.

The cleaning solution 151 removes the substance non-specifically adsorbed to the magnetic particles P. As the cleaning solution 151, water or a buffer solution, an organic solvent such as ethanol and isopropyl alcohol, or the like can be used. In a case where the buffer solution is used as the cleaning solution, salt is not particularly limited, but salt of tris or phosphoric acid is preferably used. In addition, in order to suppress the elution of RNA in the cleaning step, the surfactant such as sodium dodecyl sulfate, Triton X-100, or the like may be contained.

The PCR solution 152 is a solution for performing a process for amplifying nucleic acid by PCR. The PCR solution 152 contains, for example, reverse transcriptase, dNTP in which four kinds of deoxyribonucleotide triphosphates are mixed, and a primer for reverse transcriptase. Transcriptase is an enzyme that synthesizes complementary deoxyribonucleic acid (cDNA) using a base sequence of RNA as a template.

The chromatographic carrier accommodation portion 125 accommodates the chromatographic carrier 128. In the chromatographic carrier accommodation portion 125, the development solution 153 containing the amplified nucleic acid is developed. The chromatographic carrier 128 is a nucleic acid chromatographic carrier and indicates whether or not the target nucleic acid is present in the development solution 153.

The dispenser 106 comprises the syringes 160 to 163 for adding various liquids 150 to 153 to the respective accommodation portions 120 to 123 of the test container 101.

The pressing machine 50 comprises a plunger 52 is configured to be able to press a region corresponding to the PCR chamber 122 of the container main body 110 (here, the upper lid member 114) by the plunger 52.

The magnetic field generation and movement unit 107 includes the magnet M and a movement mechanism 170 that moves the magnet M.

The magnet M is, for example, a permanent magnet, but may be an electromagnet. As shown in FIG. 24, the magnet M is freely moved between positions A0 to A5 of the test container 101 on the upper lid member 114. The positions A0, A3, and A5 are positions where a magnetic force does not act on the magnetic particles P accommodated in the test container 101, even in a case where the magnet M is disposed. The position A1 is a position on the magnetism collecting chamber 120 and is a position where a magnetic force acts on the magnetic particles P in the magnetism collecting chamber 120 in a case where the magnet M is disposed. The position A2 is a position on the cleaning chamber 121 and is a position where magnetic force acts on the magnetic particles P in the cleaning chamber 121 in a case where the magnet M is disposed. The position A4 is a position on the PCR chamber 122 and is a position where a magnetic force acts on the magnetic particles P in the PCR chamber 122 in a case where the magnet M is disposed.

In a case of moving the magnetic particles P from the magnetism collecting chamber 120 to the cleaning chamber 121, first, the magnet M is disposed at the position A1. In a case where the magnet M is disposed at the position A1, the magnetic particles P accommodated in the magnetism collecting chamber 120 are collected by the magnetic force of the magnet M and are attracted and collected at the position corresponding to the magnet M with the upper lid member 14 interposed therebetween. In a case where the magnet M is moved to the position A2 along the upper lid member 14 from this state, the magnetic particles P are separated from the specimen solution 150 and moved to the cleaning chamber 121 according to the movement of the magnet M. Then, in a case where the magnet M is moved to the position A3, the magnetic particles P are dispersed in the cleaning solution 151.

In the same manner, in a case of moving the magnetic particles P from the cleaning chamber 121 to the PCR chamber 122, first, the magnet M is disposed at the position A2. In a case where the magnet M is disposed at the position A2, the magnetic particles P accommodated in the cleaning chamber 121 are attracted and collected at the position corresponding to the magnet M with the upper lid member 14 interposed therebetween. In a case where the magnet M is moved to the position A4 along the upper lid member 14 from this state, the magnetic particles P are separated from the cleaning solution 151 and moved to the PCR chamber 122 along the movement of the magnet M. After that, in a case where the magnet M is moved to the position A5, the magnetic particles P are dispersed in the PCR solution 152.

The movement mechanism 170 has a function of allowing the magnet M to pass the upper portion of the flow path 130 from the position A1 on the magnetism collecting chamber 120, to pass the upper portion of the flow path 131 from the position A2 on the cleaning chamber 121, and to freely move to the position A4 on the PCR chamber 122. In addition, the movement mechanism 170 moves the magnet M to the positions A0, A3 and A5 where the magnetic force does not reach the inside of the chambers 120, 121 and 122.

The nucleic acid extraction test device 100 further comprises a temperature control unit 108 (see FIG. 24). The temperature control unit 108 controls a temperature of the PCR solution in the PCR chamber 122. The temperature control unit 108 comprises a heating unit such as a heater or a Peltier element for heating a solution, and a cooling unit such as a Peltier element, a fan, a heat sink, or a liquid cooling mechanism for cooling a solution. The temperature control unit 108 raises or lowers the temperature of the solution so that the temperature is adjusted to a suitable temperature in each step of a heat denaturation step, an annealing step, and an extension step in PCR.

A transportation unit 102 is a device that relatively moves the test container 101 relatively to the dispenser 106, the magnetic field generation and movement unit 107, and the pressing machine 50. The transportation unit 102 may transport only the test container 101, or move the respective positions of the dispenser 106, the magnetic field generation and movement unit 107, and the pressing machine 50 with respect to the test container 101.

Nucleic Acid Extraction Test Method

The steps of the nucleic acid extraction test in the nucleic acid extraction test device 100 comprising the test container 101 will be described.

Pretreatment (Adsorption Process)

A sample containing RNA is mixed with a solution containing a surfactant that dissolves a cell membrane and the magnetic particles P to adsorb the RNA to the magnetic particles P. The sample containing RNA is not particularly limited, as long as it contains the RNA such as a biological sample and virus. As necessary, impurities may be removed with a filter or the like.

Magnetization Collection Process

The specimen solution 150 containing the magnetic particles P having RNA adsorbed, which was obtained in the pretreatment, is injected into the magnetism collecting chamber 120 by the syringe 160. After that, the magnet M is set at the position A1 on the magnetism collecting chamber 120. Accordingly, the magnetic particles P accommodated in the magnetism collecting chamber 120 are attracted to the magnet M and are collected at a position corresponding to the magnet M on the upper surface to be in an aggregated state (see FIG. 24).

In the magnetism collecting chamber 120, the adsorption process and the magnetism collection process may be performed in time series.

Then, by moving the magnet M along the flow path 130, the magnetic particles P are separated from the specimen solution 150 and moved to the cleaning chamber 121.

Cleaning Step

In the cleaning chamber 121, the magnetic particles P adsorbed with RNA are cleaned with the cleaning solution 151 accommodated in the cleaning chamber 121. The cleaning chamber 121 may be filled with the cleaning solution 151 in advance, or the cleaning solution 151 may be injected after the magnetic particles P are moved. The magnet M is moved to the position (position A3) where the magnetic force does not affect the cleaning chamber 121 and the magnetic particles P are dispersed in the cleaning solution 151, thereby promoting the cleaning. By performing the cleaning, the substances other than RNA that are non-specifically bound to the magnetic particles P are removed.

Then, by returning the magnet M to the position A2 on the cleaning chamber 121, the magnetic particles P are collected again at the position corresponding to the magnet M on the upper surface, and the magnet M is moved to the position A4 on the PCR chamber 122 along the flow path 131. Thereby the magnetic particles P are separated from the cleaning solution 151 and are moved to the PCR chamber 122. After that, the magnet M is moved to the position A5 where the magnetic force does not affect the PCR chamber 122, so that the magnetic particles P are dispersed in the PCR solution 152.

PCR Process

In the PCR chamber 122, the RNA adsorbed to the magnetic particles P is eluted into the PCR solution 152, and the DNA amplification by PCR is performed. The cDNA is synthesized from the extracted RNA and the cDNA is amplified by PCR. In this case, the magnetic particles P sink to the inner bottom surface of the PCR chamber 122 due to gravity.

Liquid Feeding Process

After the PCR step, the solution containing the amplified cDNA in the PCR chamber 122 is fed to the detection chamber 123. The test container 101 comprises the flow path 131 at the upper end position of the cleaning chamber 121 and the PCR chamber 122, and the flow path 132 at the upper end position of the PCR chamber 122 and the detection chamber 123, respectively. Accordingly, it is possible to prevent the passage of the solution 152 from the PCR chamber 122 to the flow paths 131 and 132 due to a capillary phenomenon or the like, before this liquid feeding process.

As shown in FIG. 25, in a case where the liquid is fed, the plunger 52 is positioned on the PCR chamber 122 and the plunger 52 is pushed down along the cylinder 54. The flexible upper lid member 114 is pushed by the plunger 52 and pushed inwards of the PCR chamber 122. This reduces the volume of the PCR chamber 122, so that the liquid in the PCR chamber 122 is fed to the detection chamber 123 through the flow path. In this case, since the return prevention structure is provided, most of the solution 152 in the PCR chamber 122 does not flow backward to the cleaning chamber 121 side, and a large amount of the solution extruded from the PCR chamber 122 can be fed to the detection chamber 123. In addition, since the flow path 132 is comprised at the upper end position of the PCR chamber 122, a supernatant portion of the PCR solution can be preferentially fed while the magnetic particles P are submerged on the inner bottom surface, and the magnetic particles P can be suppressed from flowing out to the detection chamber 123 side. By suppressing the magnetic particles P from flowing to the detection chamber 123, it is possible to perform a test with less noise in the next step.

Detection Process

In the detection chamber 123, the solution containing cDNA is mixed with the development solution. After that, the mixed liquid passes through the flow path 135 and is developed by the nucleic acid chromatographic carrier (chromatographic carrier 128) disposed in the chromatographic carrier accommodation portion 125. In a case where the RNA to be tested is contained, a positive result is obtained, and in a case where not, a negative result is obtained.

The nucleic acid extraction test is performed as described above.

Hereinabove, the case where the reverse transcription PCR method is used as the amplification method has been described, but the amplification method is not limited to the reverse transcription PCR method, and well-known amplification methods such as the transcription PCR method, the isothermal amplification method (for example, Nucleic Acid Sequence-Based Amplification (NASBA), Loop-mediated Isothermal Amplification (LAMP), transcription-reverse transcription concerted (TRC), and the like) can be used. In addition, hereinabove, the case where the nucleic acid chromatography method is used as the detection method has been described above, but the detection method is not limited to the nucleic acid chromatography method, and well-known methods such as a fluorescence detection method (intercalator method, probe method, or the like), a light scattering method using gold nanoparticles, a sequence method, an electrochemical method, a piezoelectric method, and detection of a weight or a mechanical change can be used. In these cases, the container does not necessarily comprise the chromatographic carrier 128 and the accommodation portion 125 thereof. On the other hand, the test device may comprise a detection unit suitable for various detection methods of a fluorescence detection unit and the like for detecting fluorescence from the detection chamber 123. However, the nucleic acid chromatography method is preferable because a high-priced detection system and detection equipment are not necessary and the operation in the analysis is simple.

By using the test container 101, the solution containing the DNA amplified in the PCR chamber 122 can be efficiently fed to the detection chamber 123 while suppressing the backflow to the cleaning chamber 121, and a sufficient amount of solution to be fed can be realized. Since the backflow can be suppressed to increase the amount of liquid to be fed to the detection chamber 123, a total amount of DNA that flows into the detection chamber 123 can be increased, which leads to improvement in determination accuracy.

In regard to the test container 101, a set of the test container 101, the magnetic particles P, and various treatment liquids such as the cleaning solution 151, the PCR solution 152, and the development solution 153 can also be provided as a test kit. The test kit may further include other treatment liquid such as a nucleic acid eluate. In addition, as the test kit, it is also possible to provide a set of only the test container 101 and the magnetic particles P. The magnetic particles P may be set in the magnetism collecting chamber 120 of the test container 101 in advance, or may be separately prepared.

The technology of the present disclosure is not limited to the embodiment described above, and various modifications, changes, and improvements can be made without departing from the spirit of the invention. For example, the modification examples described above may be appropriately configured in combination.

### EXAMPLES

Hereinafter, more specific examples and comparative examples of the technology of the present disclosure will be described.

Test containers A to D having the same shape as the test container 201 comprising the three accommodation portions and the flow paths connecting the three accommodation portions shown in FIGS. 1 to 4 were made, and the evaluation of the liquid feeding method was performed with respect to the examples and the comparative examples in which the pressing point and the pressing method in a case of feeding the liquid were changed. The test containers A to D (test container 201) comprise the first accommodation portion 221, the second accommodation portion 222, the third accommodation portion 223, the first flow path 231 connecting the first accommodation portion 221 and the second accommodation portion 222 at the upper end, and the second flow path 232 connecting the second accommodation portion 222 and the third accommodation portion 223 at the upper end. The three accommodation portions 221, 222, and 223 had the same shape, and had a length L of 7.5 mm, a width W of 7.5 mm, and a depth d of 1 mm. A width of the first flow path 231 and the second flow path 232 was 1 mm. In addition, a height h of each of the first flow path 231 and the second flow path 232 from the inner bottom surface 222a of the second accommodation portion 222 was 0.8 mm.

First, the test containers A to D were manufactured as follows.

Test Container A

The same test container A was used in Examples 1 to 6 and Comparative Examples 1 and 2.

The test container A was formed of the container main body 212 and the upper lid member 214, and the container main body 212 was formed of a main body portion forming the side wall surfaces of the first accommodation portion 221, the second accommodation portion 222, and the flow path 230, and a bottom surface member forming the inner bottom surfaces of the first accommodation portion 221, the second accommodation portion 222, and the flow path 230.

Polycarbonate (PC) was used as the material of the main body portion 210. Specifically, the main body portion was injection-molded using IUPILON EB-3001R manufactured by Mitsubishi Engineering Plastics Co., Ltd. As the bottom surface member, Technoloy C000 (thickness of 100 μm) manufactured by Sumika Acrylic Sales Co., Ltd. was used. In addition, a silicone film GFSX6000 (thickness of 300 μm) manufactured by Tomita Mateqs Co., Ltd. was used for the upper lid member 214.

The bottom surface member was roller-bonded to the bottom surface of the main body portion using an adhesive #9969 manufactured by 3M Japan Co., Ltd., and the upper lid member 214 was roller-bonded to the upper surface of the main body portion using a silicone adhesive NSD-50 manufactured by Nipper Co., Ltd. to obtain a test container A.

Test Container B

A test container B was used in Example 7. The test container B had the same shape as the test container A, except that polypropylene (PP) was used as the material of the main body 210.

The main body portion was injection-molded using WINTEC WMG03UX manufactured by Japan Polypro Corporation. As the bottom surface member, Trefan BO60-2500 (thickness of 60 μm) manufactured by Toray Industries, Inc. was used. In addition, in the same manner as in Example 1, a silicone film GFSX6000 (thickness of 300 μm) manufactured by Tomita Mateqs Co., Ltd. was used for the upper lid member 214.

Test Container C

A test container C was used in Example 8. The test container C had the same shape as the test container A, except that polymethyl methacrylate resin (PMMA) was used as the material of the main body 210.

The main body portion was injection-molded using Acrypet VH001 manufactured by Mitsubishi Chemical Corporation, and Technoloy 5001 manufactured by Sumika Acrylic Sales Co., Ltd. (thickness 125 μm) was used as the bottom surface member. In addition, in the same manner as in Example 1, a silicone film GFSX6000 (thickness of 300 μm) manufactured by Tomita Mateqs Co., Ltd. was used for the upper lid member 214.

Test Container D

A test container D was used in Example 9. The test container D had the same shape as the test container A, except that COP was used as the material of the main body 210.

The main body portion was injection-molded using ARTON F4520 manufactured by JSR Corporation, and a film having a thickness of 50 μm obtained by forming a film of ARTON R5000 manufactured by JSR Corporation was used as the bottom surface member. In addition, in the same manner as in Example 1, a silicone film GFSX6000 (thickness of 300 μm) manufactured by Tomita Mateqs Co., Ltd. was used for the upper lid member 214.

Evaluation of Liquid Feeding Properties

Using the test containers A to D, liquid feeding was performed by the methods of Examples 1 to 9 and Comparative Examples 1 and 2 below, and the liquid feeding properties were evaluated.

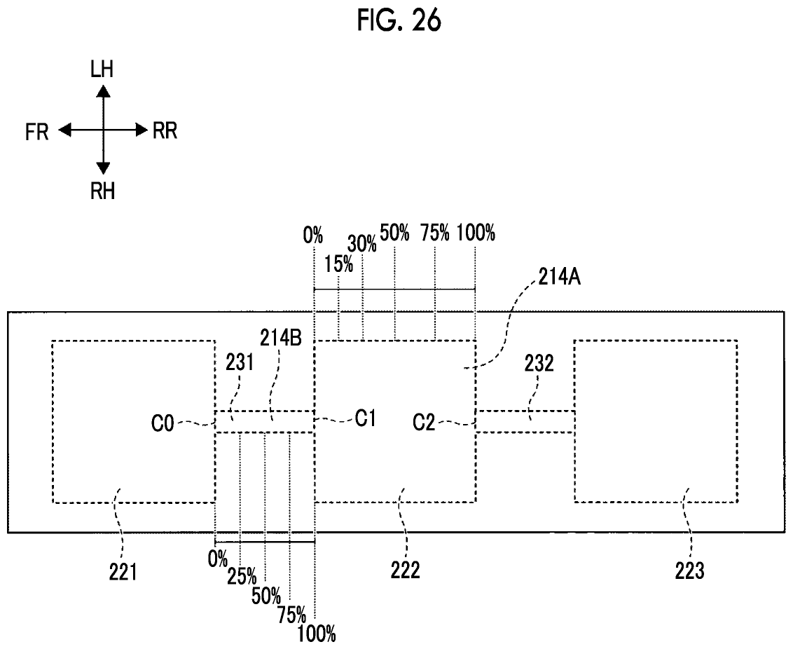

As shown in FIG. 26, a pushing position of the portion 214A on the upper portion of the first accommodation portion 222 is shown as a position in a case where the boundary C1 between the second accommodation portion 222 and the first flow path 231 is 0% position and the boundary C2 between the second accommodation portion 222 and the third flow path 232 is 100% position. In addition, the pushing position of the portion 214B on the upper portion of the first flow path 231 is shown as a position in a case where the boundary C0 between the first flow path 231 and the first accommodation portion 221 is 0% position and the boundary C1 between the first flow path 231 and the second accommodation portion 222 is 100% position.

After filling the second accommodation portion 222 with water, the liquid was fed by the method of each example and comparative example, a weight of the liquid fed to the third accommodation portion 223 and a weight of the liquid flowed back to the first accommodation portion 221 were measured, and a ratio thereof was calculated as a liquid return rate.

That is,

Liquid return rate=(Weight of liquid returned to the first accommodation portion [mg])/(Weight of liquid fed to the third accommodation portion [mg]).

The liquid return rate was evaluated according to the following criteria. For practical use, E or higher is required. In addition, practically, D or higher is preferable, C or higher is more preferable, and B or higher is further preferable.

A: less than 5%

B: 5% or more and less than 10%

C: 10% or more and less than 15%

D: 15% or more and less than 20%

E: 20% or more and less than 25%

F: 30% or more

### Example 1

Using the test container A, a 30% position of the portion 214A on the second accommodation portion 222 was pressed by a ball plunger to carry out liquid feeding (see FIG. 5).

### Example 2

Using the test container A, a 15% position of the portion 214A on the second accommodation portion 222 was pressed by a ball plunger to carry out liquid feeding (see FIG. 5).

### Example 3

Using the test container A, a 25% position of the portion 214B on the first flow path 231 and a 50% position of the portion 214A on the second accommodation portion 222 were pressed by a ball plunger to carry out liquid feeding (see FIG. 7). In this case, the portions 214A and 214B were pressed at the same time.

### Example 4

Using the test container A, a 50% position of the portion 214B on the first flow path 231 and a 50% position of the portion 214A on the second accommodation portion 222 were pressed by a ball plunger to carry out liquid feeding (see FIG. 7). In this case, the portions 214A and 214B were pressed at the same time.

### Example 5

Using the test container A, a 75% position of the portion 214B on the first flow path 231 and a 50% position of the portion 214A on the second accommodation portion 222 were pressed by a ball plunger to carry out liquid feeding (see FIG. 7). In this case, the portion 214B on the first flow path was pressed in first, and then the portion 214A on the second accommodation portion 222 was pressed in.

### Example 6

Using the test container A, a 50% position of the portion 214B on the first flow path 231 and a 50% position of the portion 214A on the second accommodation portion 222 were pressed by a ball plunger to carry out liquid feeding (see FIG. 7). In this case, the portions 214A and 214B were pressed at the same time.

### Example 7

Using the test container B, a 30% position of the portion 214A on the second accommodation portion 222 was pressed by a ball plunger to carry out liquid feeding (see FIG. 5).

### Example 8

Using the test container C, a 30% position of the portion 214A on the second accommodation portion 222 was pressed by a ball plunger to carry out liquid feeding (see FIG. 5).

### Example 9

Using the test container D, a 30% position of the portion 214A on the second accommodation portion 222 was pressed by a ball plunger to carry out liquid feeding (see FIG. 5).

### Comparative Example 1

Using the test container A, a 50% position of the portion 214A on the second accommodation portion 222 was pressed by a ball plunger to carry out liquid feeding.

### Comparative Example 2

Using the test container A, a 75% position of the portion 214A on the second accommodation portion 222 was pressed by a ball plunger to carry out liquid feeding.

Table 1 collectively shows the container, the liquid feeding method, and the evaluation result of each example.

TABLE 1

| | | Liquid feeding method | | | |
|---|---|---|---|---|---|
| | | | | Second accommodation | Evaluation |
| | | | First flow path | portion | Liquid feeding |
| | | Pressing method | Pressing position | Pressing position | properties |
| Example 1 | Test container A | Only second accommodation portion | | 30% position | E |
| Example 2 | Test container A | Only second accommodation portion | | 15% position | D |
| Example 3 | Test container A | Pressing first flow path and second | 25% position | 50% position | B |
| | | accommodation portion at the same | | | |
| | | time | | | |
| Example 4 | Test container A | Pressing first flow path and second | 50% position | 50% position | A |
| | | accommodation portion at the same | | | |
| | | time | | | |
| Example 5 | Test container A | Pressing first flow path and second | 75% position | 50% position | A |
| | | accommodation portion at the same | | | |
| | | time | | | |
| Example 6 | Test container A | Pressing first flow path and second | 25% position | 50% position | A |
| | | accommodation portion in this order | | | |
| Example 7 | Test container B | Only second accommodation portion | | 30% position | E |
| Example 8 | Test container C | Only second accommodation portion | | 30% position | E |
| Example 9 | Test container D | Only second accommodation portion | | 30% position | E |
| Comparative | Test container A | Only second accommodation portion | | 50% position | F |
| Example 1 | | | | | |
| Comparative | Test container A | Only second accommodation portion | | 75% position | F |
| Example 2 | | | | | |

As shown in Table 1, as shown in Examples 1 and 2, by pressing the position of the second accommodation portion 222 closer to the first flow path 231, it was possible to improve the liquid feeding properties to the third accommodation portion 223 side, compared to Comparative Examples 1 and 2 in which the central portion of the second accommodation portion 222 (50% position) and the position closer to the second flow path 232 (75% position) were pressed.

In Examples 1 and 7 to 9 in which, while the main body portions of the test containers have different formation materials, but have the same shape, and the same position is pressed, the same results were obtained. It is considered that the liquid feeding properties of the technology of the present disclosure do not depend on the material of the main body portion, and the same results can be obtained.

As in Examples 1 and 2, in a case where the liquid is fed with a configuration in which the position of the portion 214A forming the upper wall surface of the second accommodation portion 222 closer to the first flow path 231 is pressed by using one plunger (configuration of liquid feeding device of the first embodiment), the liquid feeding properties were higher in a case where the pressing position is closer to the first flow path.

As in Examples 3 to 6, in a case where the liquid is fed with a configuration in which the two portions of the first portion 214B forming the upper wall surface of the first flow path 231 and the second portion 214A forming the upper wall surface of the second accommodation portion 222 are pressed (configuration of liquid feeding device of the second embodiment) by using the two plungers, the liquid feeding properties were higher than in Examples 1 and 2, and more excellent effect of preventing the backflow to the first flow path 231 was obtained. It was found that it is particularly preferable that the pressing position of the first flow path 231 includes the center position (50% position) in the flow path length direction on the second accommodation portion 222 side, in order to improve the liquid feeding properties.

### EXPLANATION OF REFERENCES

1, 1A, 2, 3, 3A, 4, 5, 6, 211: Test container
10, 10A, 10B, 10C, 10D, 10E, 10F, 10G: Container main body
12, 12A, 12B, 12C, 12D, 12E, 12F, 12G: Main body portion
14: Upper lid member
14A: Portion (first part)
14B: Second portion
21: First accommodation portion
21b: Upper wall surface of first accommodation portion
22: Second accommodation portion
22a: Inner bottom surface of second accommodation portion
22b: upper wall surface of second accommodation portion
22c: Inner side surface of second accommodation portion
23: Third accommodation portion
31: First flow path
31a: Inner bottom surface of first flow path
32: Second flow path
32a: Inner bottom surface of second flow path
33, 43: Corner
34: Hydrophobic surface
40: Stepped portion
41, 42: Step
50, 55: Pressing machine
51, 52: Plunger
53, 54: Cylinder
100: nucleic acid extraction test device
101: test container
102: Transportation unit
106: Dispenser
107: Magnetic field generation and movement unit
108: Temperature control unit
110: Container main body
112: Main body portion
114: Upper lid member
120: Magnetism collecting chamber (accommodation portion)
121: Cleaning chamber (first accommodation portion)
122: PCR chamber (second accommodation portion)
122a: Inner bottom surface of PCR chamber
123: Detection chamber (third accommodation portion)
125: Chromatographic carrier accommodation portion
128: Chromatographic carrier
130, 131, 132, 135, 145: flow path
150: specimen solution
151: Cleaning solution
152: PCR solution
153: Development solution
160-163: Syringe
170: Movement mechanism
201: Liquid feeding device
210: Container main body
211: Test container
212: Main body portion
214: Upper lid member
221: First accommodation portion
222: Second accommodation portion
223: Third accommodation portion
231: First flow path
232: Second flow path
240: Stepped portion
241, 242, 243: step
M: Magnet
P: Magnetic particles

What is claimed is:

1. A liquid feeding device, comprising:
a test container including a first accommodation portion, a second accommodation portion, and a third accommodation portion each accommodating a liquid, a first flow path connecting the first accommodation portion and the second accommodation portion to each other at respective upper end positions thereof, and a second flow path connecting the second accommodation portion and the third accommodation portion to each other at respective upper end positions thereof, in which at least a portion forming an upper wall surface of the second accommodation portion has flexibility; and
a pressing machine which includes a pressing portion and presses the portion forming the upper wall surface of the second accommodation portion at a position closer to the first flow path from a center in a liquid feeding direction towards an inside of the second accommodation portion from an outside by the pressing portion,
wherein the liquid accommodated in the second accommodation portion is fed to the third accommodation portion by pressing the portion forming the upper wall surface of the second accommodation portion with the pressing machine.

2. A liquid feeding device, comprising:
a test container including a first accommodation portion, a second accommodation portion, and a third accommodation portion each accommodating a liquid, a first flow path connecting the first accommodation portion and the second accommodation portion to each other at respective upper end positions thereof, and a second flow path connecting the second accommodation portion and the third accommodation portion to each other at respective upper end positions thereof, in which at least a first portion forming an upper wall surface of the first flow path and a second portion forming an upper wall surface of the second accommodation portion have flexibility; and
a pressing machine which includes a first pressing portion and a second pressing portion, presses the first portion forming the upper wall surface of the first flow path towards the inside of the first flow path from outside by the first pressing portion, and presses the second portion having flexibility of the upper wall surface of the second accommodation portion towards an inside of the second accommodation portion from an outside by the second pressing portion,
wherein the liquid accommodated in the second accommodation portion is fed to the third accommodation portion by pressing the first portion and the second portion with the pressing machine.

3. The liquid feeding device according to claim 2,
wherein the pressing machine simultaneously performs a pressing operation by the first pressing portion and a pressing operation by the second pressing portion.

4. The liquid feeding device according to claim 2,
wherein the pressing machine performs a pressing operation by the first pressing portion before a pressing operation by the second pressing portion, and performs the pressing operation by the second pressing portion while maintaining a pressing state by the first pressing portion.

5. The liquid feeding device according to claim 1,
wherein the test container includes a main body portion in which a portion forming each of the first accommodation portion, the first flow path, the second accommodation portion, the second flow path, and the third accommodation portion is open, and an upper lid member including the portion forming the upper wall surface of the second accommodation portion, and the first accommodation portion, the first flow path, the second accommodation portion, the second flow path, and the third accommodation portion are internally formed by covering an opening of the main body portion with the upper lid member.

6. The liquid feeding device according to claim 2,
wherein the test container includes a main body portion in which a portion forming each of the first accommodation portion, the first flow path, the second accommodation portion, the second flow path, and the third accommodation portion is open, and an upper lid member including the portion forming the upper wall surface of the second accommodation portion, and the first accommodation portion, the first flow path, the second accommodation portion, the second flow path, and the third accommodation portion are internally formed by covering an opening of the main body portion with the upper lid member.

7. The liquid feeding device according to claim 5,
wherein the upper lid member of the test container has flexibility over an entire area.

8. The liquid feeding device according to claim 6,
wherein the upper lid member of the test container has flexibility over an entire area.

9. The liquid feeding device according to claim 5,
wherein the test container includes a liquid return prevention structure which prevents a backflow of the liquid to the first accommodation portion, in a case where the liquid accommodated in the second accommodation portion is fed to the third accommodation portion via the second flow path.

10. The liquid feeding device according to claim 6,
wherein the test container includes a liquid return prevention structure which prevents a backflow of the liquid to the first accommodation portion, in a case where the liquid accommodated in the second accommodation portion is fed to the third accommodation portion via the second flow path.

11. The liquid feeding device according to claim 9,
wherein the liquid return prevention structure of the test container has a structure in which a height from an inner bottom surface of the second accommodation portion to an inner bottom surface of the first flow path is higher than a height from the inner bottom surface of the second accommodation portion to an inner bottom surface of the second flow path.

12. The liquid feeding device according to claim 9,
wherein the liquid return prevention structure of the test container has a structure of the first flow path and the second flow path in which a water contact angle of an inner surface of the first flow path is set to be greater than a water contact angle of an inner surface of the second flow path.

13. The liquid feeding device according to claim 10,
wherein the liquid return prevention structure of the test container has a structure of the first flow path and the second flow path in which a water contact angle of an inner surface of the first flow path is set to be greater than a water contact angle of an inner surface of the second flow path.

14. The liquid feeding device according to claim 9,
wherein the liquid return prevention structure of the test container has a structure of a stepped portion which is provided between the first flow path and the second accommodation portion and which includes two or more steps from an inner bottom surface of the first accommodation portion.

15. The liquid feeding device according to claim 10,
wherein the liquid return prevention structure of the test container has a structure of a stepped portion which is provided between the first flow path and the second accommodation portion and which includes two or more steps from an inner bottom surface of the first accommodation portion.

16. The liquid feeding device according to claim 1,
wherein the test container further comprises a chromatographic carrier for performing a nucleic acid test, and a carrier accommodation portion accommodating the chromatographic carrier.

17. The liquid feeding device according to claim 2,
wherein the test container further comprises a chromatographic carrier for performing a nucleic acid test, and a carrier accommodation portion accommodating the chromatographic carrier.

18. The liquid feeding device according to claim 1,
wherein, in the test container, the first accommodation portion accommodates a first liquid containing magnetic particles, the first flow path allows separated magnetic particles separated from the first liquid to pass through the first flow path, and the second accommodation portion accommodates the separated magnetic particles.

19. The liquid feeding device according to claim 2,
wherein, in the test container, the first accommodation portion accommodates a first liquid containing magnetic particles, the first flow path allows separated magnetic particles separated from the first liquid to pass through the first flow path, and the second accommodation portion accommodates the separated magnetic particles.

20. The liquid feeding device according to claim 18, further comprising:
a magnetic field generation and movement unit including a magnet and a movement mechanism which moves the magnet,
wherein the magnet is disposed on the first accommodation portion of the test container to collect the magnet particles in the liquid and the magnet is moved onto the second accommodation portion from the first accommodation portion along the first flow path, so that the separated magnetic particles separated from the first liquid passes through the first flow path to be moved to the second accommodation portion.
